(19) United States
(12) Patent Application Publication
(10) Pub. No.: US 2026/0092925 A1
(43) Pub. Date: Apr. 2, 2026

(54) IMPROVED FLUORESCENT PROTEINS

(71) Applicant: NUCLERA LTD, Cambridge, Cambridgeshire (GB)

(72) Inventor: Florian Mittelberger, Cambridge (GB)

(21) Appl. No.: 18/878,983

(22) PCT Filed: Jul. 11, 2023
(86) PCT No.: PCT/GB2023/051817

§ 371 Date: Dec. 26, 2024

(30) Foreign Application Priority Data
Jul. 11, 2022 (GB) 2210173.7
Feb. 23, 2023 (WO) PCT/GB2023/050415

(51) Int. Cl. G01N 33/68; C07K 14/435; G01N 33/58

(52) U.S. Cl. CPC G01N 33/68; C07K 14/43595; G01N 33/582

(57) ABSTRACT

Provided herein are methods and compositions for the on-device detection of protein synthesis using fluorescent proteins. The methods are applicable to monitoring on a microfluidic device.

### CROSS REFERENCE TO RELATED APPLICATIONS

This application is a U.S. National Stage Application filed under 35 U.S.C. § 371, based on International Patent Application No. PCT/GB2023/051817, filed on Jul. 11, 2023, which claims priority to UK Patent Application No. GB2210173.7, filed on Jul. 11, 2022, and International Application No. PCT/GB2023/050415, filed Feb. 23, 2023. The entire contents of each of the above applications are incorporated herein by reference.

### STATEMENT REGARDING SEQUENCE LISTING

The instant application contains a Sequence Listing XML file, which has been submitted electronically in .XML format. Said Sequence Listing XML file, created on Dec. 4, 2025, is named P077 K45E Sequence Listing Updated 251204.xml, and is 88,378 bytes in size. The Sequence Listing XML file is part of the specification and is hereby incorporated by reference herein in its entirety.

### FIELD OF THE INVENTION

Provided herein are methods and compositions for the on-device detection of protein synthesis using fluorescent proteins. The methods are applicable to monitoring on a microfluidic device.

### BACKGROUND TO THE INVENTION

When performing cell-free protein synthesis at microfluidic scale in a microfluidic device, such as a digital microfluidic device, it is useful to detect in real-time the proteins that are synthesized from said cell-free protein synthesis reaction. However, it is difficult to perform real-time detection of proteins in a cell-free protein synthesis reaction environment. The reaction contains many other proteins and biomolecules at high concentration, making non-specific protein detection via standard protein staining methods difficult (e.g., Coomassie Brilliant Blue G-250, SYPRO™ Ruby, Silver staining). Immunostaining or affinity-based purification followed by non-specific proteins staining are equally unhelpful as significant washing on a solid support must be performed to prevent background interference. As washing is known to be difficult in microfluidic and digital microfluidic devices, background interference may become debilitating.

Currently existing luminescent complementation approaches cannot achieve prolonged real-time detection in cell-free protein synthesis reactions, which often extend beyond 16 hours. This limit is due to a combination of reasons including O₂ consumption by the luminescence-generating enzyme that competes with cell-free protein synthesis O₂ requirements and temporary or permanent exhaustion of luminescent substrate over 3-24 hours of recombinant protein expression detection.

Proteins of interest may also be expressed as a fusion to a fluorescent protein, such as green fluorescent protein (GFP). However, GFP is a 26.9 kDa protein, which is the typical size for most fluorescent proteins. Tags of this size increase the total size of the protein of interest, especially if the protein of interest must be tagged with other large fusion proteins such as maltose-binding protein (MBP), which is 42.5 kDa. Given the average size of a human protein is ˜52 kDa and the average size of an E. coli protein is ˜35 kDa (Kim, Y. E. et al. Annu. Rev. Biochem. 2013. 82:323-355), the addition of a comparably sized fluorescent protein tag can significantly change the biological function and biophysical characteristics of a protein.

Many pieces of prior art disclose the use of sub-component tags for monitoring expression in a cellular system. For example U.S. Pat. No. 7,666,606 discloses protein-protein interaction detection systems using microdomains.

Schinn et al. Biotechnol. Bioeng 114 10 Oct. 2017 2412-2417. (https://onlinelibrary.wiley.com/doi/10.1002/bit.26305) discloses Rapid in-vitro screening for the location-dependent effects of unnatural amino acids on protein expression and activity-Schinn—2017—Biotechnology and Bioengineering—Wiley Online Library.

Split green fluorescent protein (GFP) has been used in a variety of applications. ccGFP is a green fluorescent protein engineered from a tetrameric GFP found in Corynactis californica, a bright red colonial anthozoan similar to sea anemones and scleractinian stony corals, as described in Nguyen et al; Nature Scientific Reports volume 11, Article number: 18440 (2021). The inventors herein have optimised the use of ccGFP for use in cell-free protein synthesis.

### SUMMARY

The split ccGFP variant can be used as a general method for the detection of proteins of interest (POI) that include the ccGFP₁₁ v1 peptide at either terminus of their coding sequence. The disadvantage of using previously described split ccGFP systems is the low solubility of the proteins, making it necessary to purify the protein from inclusion bodies and limiting the working concentration available for detection assays.

The invention herein describes an improved ccGFP variant amino acid sequence and components thereof, nucleic acid sequences for the expression thereof and uses of the improved fluorescent protein. Mutation of the strands adjacent to strand 11 can be modified in order to improve the properties of the protein. Beta-barrel strands 3 and 10 are within H-bonding interaction with strand 11. The changes should not destabilise the GFP₁₋₁₀:GFP₁₁ complementation, but can alter the exposed hydrophobic core of the split GFP, which is likely to be the cause of insoluble expression.

The ccGFP₁₋₁₁ wild type amino acid sequence is 221 amino acids (Strand 3 underlined):

(SEQ ID NO: 1)
MSLSKQVVKEDMKMTYHMDGCVNGHYFTIEGEGTGKPFKGQKTLK
LRVTEGGPLPFAFDILSATFTYGNRCFCDYPEDMPDYFKQSLPEG
YSWERTMMYEDGACGTASAHISLDKNGFVHNSTFHGVNFPANGPV
MKKKGVNWEPSSEKITACDGILKGDVTMFLVLEGGHRLKCLFQTT
YKADKVVKMPPNHIIEHRLVRSEDGDAVQIQEHAVAKYFTV

Disclosed is a ccGFP₁₋₁₁ sequence having a mutation in the strand 3 region GQKTLKLRV (residues 40-48). Disclosed is a ccGFP₁₋₁₁ sequence having a mutation at position K45, which may be K45E. Disclosed is an amino acid sequence having the position 45E having at least 90% homology to the sequence:

(SEQ ID NO: 2)
MSLSKQVVKEDMKMTYHMDGCVNGHYFTIEGEGTGKPFKGQKTLE
LRVTEGGPLPFAFDILSATFTYGNRCFCDYPEDMPDYFKQSLPEG
YSWERTMMYEDGACGTASAHISLDKNGFVHNSTFHGVNFPANGPV
MKKKGVNWEPSSEKITACDGILKGDVTMFLVLEGGHRLKCLFQTT
YKADKVVKMPPNHIIEHRLVRSEDGDAVQIQEHAVAKYFTV

The sequence may be used in split form, for example as ccGFP₁₁ and ccGFP₁₋₁₀. Sequences described may be truncated such that amino acids can be removed from the N or C terminus without affecting the activity or fluorescence of the folded core. Where sequences are truncated the modified positions may change numbering, the reference to K45 refers to the wild type sequence of 221 amino acids. The reference to strand 3 refers to amino acids 40-48 of the wild type sequence of 221 amino acids.

Strand three amino acid changes yielded promising positions for amino acid changes. One example of an amino acid location is K45. This can be altered to K45E. The strand 3 sequence may comprise GEQELELRV (SEQ ID NO: 3).

Described is the particular ccGFP₁₋₁₀ variant K45E. This will, once mixed with ccGFP₁₁, bind to the ccGFP₁₁, complementing the missing beta sheet 11, resulting in a fluorescence signal that can be detected and quantified. This allows for the detection of ccGFP₁₁ tagged proteins both in-vitro and in-vivo. The here described ccGFP₁₋₁₀ K45E protein variant shows an improved solubility when compared to the original ccGFP₁₋₁₀ parent. Solubility can be further improved by the addition of a C-terminal MBP fusion protein.

The changes herein improve the physical properties of the protein (e.g. solubility; complementation rate) without influencing others (e.g. colour of the fluor; sequence of the split strand etc). By way of example; parent ccGFP₁₋₁₀ is 5-10% soluble when expressed. The mutation described herein increased this to approx. 55%. Adding MBP as a fusion protein increases the solubility even further. The parent protein (27 KDa), when concentrated over 1 mg/mL caused precipitation. With the variant described herein, the mutated protein in its elution buffer can be concentrated to 65 mg/mL without significant precipitation. This higher concentration allows direct use to perform in situ (real time and end point) detection in small volumes of liquid, rather than needing large final dilutions in order to add sufficient protein for an end point reading.

Here, we report the use of the improved split peptide system for fluorescence-based monitoring of the expression of a protein of interest in cell-free protein synthesis reactions. The monitoring can be performed in real-time as well as providing an end-point measurement.

Disclosed herein is a ccGFP variant comprising a K45E mutation. The variant can be a ccGFP₁₋₁₀ variant or a ccGFP₁₋₁₁ variant. The ccGFP₁₋₁₀ variant can be complexed with ccGFP₁₁ variant.

The ccGFP₁₋₁₀ K45E variant sequence may comprise a sequence:

(SEQ ID NO: 4)
MSMEKQVLKENMKTTYHMDGSVDGHYFEIEGEGTGNPFKGEQELE
LRVTKGGPLPFAFDILSPTFTYGNRVFTDYPEDMPDYFKQSLPEG
YSWERTMMYEDGATATASARISLDKNGFVHKSTFHGENFPANGPV
MKKKGVDWEPSSETITPEDGILKGDVEMFLVLEGGQRLKALFQTT
YKANKVVKMPPRHKIEHRLVRS

The underlined E is the site of alteration from the wild type K

```
      10 20 30 40 50 60
MSLSKQVVKEDMKMTYHMDGCVNGHYFTIEGEGTGKPFKGQKTLKLRVTEGGPLPFAFDI
:: ::: :: :: :::::: : :::: ::::::: :::: : :::: ::::::::::
MSMEKQVLKENMKTTYHMDGSVDGHYFEIEGEGTGNPFKGEQELELRVTKGGPLPFAFDI
      10 20 30 40 50 60
      70 80 90 100 110 120
LSATFTYGNRCFCDYPEDMPDYFKQSLPEGYSWERTMMYEDGACGTASAHISLDKNGFVH
:: ::::::: : :::::::::::::::::::::::::::::: :::: ::::::::::
LSPTFTYGNRVFTDYPEDMPDYFKQSLPEGYSWERTMMYEDGATATASARISLDKNGFVH
      70 80 90 100 110 120
      130 140 150 160 170 180
NSTFHGVNFPANGPVMKKKGVNWEPSSEKITACDGILKGDVTMFLVLEGGHRLKCLFQTT
::::: :::::::::::::: :::::: :: :::::::: :::::::: ::: :::::
KSTFHGENFPANGPVMKKKGVDWEPSSETITPEDGILKGDVEMFLVLEGGQRLKALFQTT
      130 140 150 160 170 180
      190 200 210 220
YKADKVVKMPPNHIIEHRLVRSEDGDAVOIQEHAVAKYFTV
::: ::::::: : ::::::::
YKANKVVKMPPRHKIEHRLVRS
```

The protein ccGFP K45E variant sequence may comprises a sequence with greater than 95% homology to sequence having the E fixed:

(SEQ ID NO: 4)
MSMEKQVLKENMKTTYHMDGSVDGHYFEIEGEGTGNPFKGEQELE
LRVTKGGPLPFAFDILSPTFTYGNRVFTDYPEDMPDYFKQSLPEG
YSWERTMMYEDGATATASARISLDKNGFVHKSTFHGENFPANGPV
MKKKGVDWEPSSETITPEDGILKGDVEMFLVLEGGQRLKALFQTT
YKANKVVKMPPRHKIEHRLVRS

or a C or N terminal truncation thereof which binds to ccGFP₁₁ to become fluorescent.

The protein is highly soluble. The mutated protein can be used at concentrations of greater than 10 mg/mL. The mutated protein can be used at concentrations of greater than 50 mg/mL. The mutated protein can be used at concentrations of greater than 65 mg/mL. The proteins can be attached to additional sequences such as maltose binding protein (MBP) to further increase solubility.

Also disclosed is a nucleic acid sequence coding for an amino acid sequence having a K45E mutation. The nucleic acid sequence may comprise:

(SEQ ID NO: 5)
ATGAGCATGGAAAAACAGGTGCTGAAAGAAAACATGAAAACCACC
TATCACATGGATGGTAGCGTTGATGGTCACTATTTTGAAATTGAA
GGTGAAGGCACCGGCAATCCGTTTAAAGGTGAACAAGAACTGGAA
CTCCGTGTTACCAAAGGTGGTCCGCTGCCGTTTGCATTTGATATT
CTGAGCCCGACCTTTACCTATGGTAATCGTGTTTTTACCGACTAT
CCGGAAGATATGCCGGATTATTTCAAACAGAGCCTGCCGGAAGGT
TATAGCTGGGAACGTACCATGATGTATGAAGATGGTGCAACCGCA
ACCGCCAGCGCACGTATTAGCCTGGATAAAAATGGTTTTGTGCAT
AAGAGCACCTTTCACGGTGAAAACTTTCCGGCAAATGGTCCGGTT
ATGAAAAAGAAAGGTGTTGATTGGGAACCGAGCAGCGAAACCATT
ACACCGGAAGATGGTATTCTGAAAGGTGATGTTGAAATGTTTCTG
GTTCTGGAAGGTGGTCAGCGTCTGAAAGCCCTGTTTCAGACCACC
TATAAAGCCAATAAAGTGGTTAAAATGCCTCCGCGTCATAAAATT
GAACATCGTCTGGTTCGTAGC.

Also disclosed is a method of detecting a protein of interest comprising taking a protein of interest attached to ccGFP₁₁, binding the ccGFP₁₁ to a ccGFP₁₋₁₀ variant K45E and monitoring the presence of the protein of interest by detecting a fluorescent signal from the assembled ccGFP₁₋₁₁. Also disclosed is the use of the ccGFP₁₋₁₀ variant K45E as a solubility tag to aid soluble expression of the protein of interest.

Disclosed herein is a method for the monitoring of cell-free protein synthesis in a droplet on a digital microfluidic device comprising
a. cell-free transcription and translation of a protein of interest fused to a ccGFP₁₁ peptide tag; and
b. monitoring the presence of the peptide tag using a further ccGFP₁₋₁₀ polypeptide having a K45E amino acid change, which in the presence of the ccGFP₁₁ peptide tag produces a detectable signal.

The use of the terms “in-vitro” and “cell-free” may be used interchangeably herein.

Any in-vitro transcription and translation may be used, for example extract-based systems derived from rabbit reticulocyte lysate, human lysate, Chinese Hamster Ovary lysate, a wheat germ lysate, HEK293 lysate, E. coli lysate, insect lysate, yeast lysate.

Alternatively the in-vitro transcription and translation may be assembled from purified components, for example a system of purified recombinant elements (PURE).

The in-vitro transcription and translation may be coupled or uncoupled.

The sequence may have a greater than 90% homology to any sequence mentioned herein. The sequence may have a greater than 95% homology to any sequence mentioned herein.

The variant sequence may contain a greater than 95% homology to sequence:

(SEQ ID NO: 6)
MSMEKQVLKENMKTTYHMDGSVDGHYFEIEGEGTGNPFKGEQELK
LRVTKGGPLPFAFDILSPTFTYGNRVFTDYPEDMPDYFKQSLPEG
YSWERTMMYEDGATATASARISLDKNGFVHKSTFHGENFPANGPV
MKKKGVDWEPSSETITPEDGILKGDVEMFLVLEGGQRLKALFQTT
YKANKVVKMPPRHKIEHRLVRS

having one or more amino acid changes.

The sequence may contain a greater than 95% homology to sequence having the E fixed:

(SEQ ID NO: 4)
MSMEKQVLKENMKTTYHMDGSVDGHYFEIEGEGTGNPFKGEQELELRVTK
GGPLPFAFDILSPTFTYGNRVFTDYPEDMPDYFKQSLPEGYSWERTMMYE
DGATATASARISLDKNGFVHKSTFHGENFPANGPVMKKKGVDWEPSSETI
TPEDGILKGDVEMFLVLEGGQRLKALFQTTYKANKVVKMPPRHKIEHRLV
RS.

The complementary GFP₁₁ peptide amino acid sequence could be the following:

(SEQ ID NO: 7)
1. KRDHMVLLEFVTAAGITGT
(SEQ ID NO: 8)
2. KRDHMVLHEFVTAAGITGT
(SEQ ID NO: 9)
3. KRDHMVLHESVNAAGIT
(SEQ ID NO: 10)
4. RDHMVLHEYVNAAGIT
(SEQ ID NO: 11)
5. GDAVQIQEHAVAKYFTV
(SEQ ID NO: 12)
6. GDTVQLQEHAVAKYFTV
(SEQ ID NO: 13)
7. GETIQLQEHAVAKYFTE

or a truncated version thereof. Truncations may involve a shortening of up to 5 amino acids from the N terminus, the C terminus or a combination thereof.

GFP₁₁ or GFP₁₋₁₀ can be fused to the protein of interest through an amino acid linker. In one embodiment, the oligopeptide, peptide, or polypeptide linker can be 0-50 amino acids.

Also disclosed are nucleic acid sequences for expressing particular tags. Nucleic acid sequences include

(SEQ ID NO: 14)
5′GGTGATACCGTTCAGCTGCAAGAACATGCAGTTGCAAAATACTTTACC
GTG
(SEQ ID NO: 15)
5′GGTGAAACCATCCAGTTACAAGAACACGCCGTGGCCAAATATTTCACC
GAA

These sequences may be repeated one or more times to produce a protein having multiple GFP₁₁ domains.

The protein may be fused to multiple tags. For example the protein may be fused to multiple ccGFP₁₁ peptide tags and the synthesis occurs in the presence of multiple ccGFP₁₋₁₀ polypeptides. The protein of interest may be fused to one or more sfCherry₁₁ peptide tags and one or more GFP₁₁ peptide tags and the synthesis occurs in the presence of one or more GFP₁₋₁₀ polypeptides and one or more sfCherry₁₋₁₀ polypeptides.

Any protein of interest may be synthesised. The protein may be an enzyme, for example a terminal deoxynucleotidyl transferase (TdT) enzyme or a truncated version thereof or the homologous amino acid sequence of a terminal deoxynucleotidyl transferase (TdT) enzyme in other species or the homologous amino acid sequence of Polμ, Polβ, Polλ, and Polθ of any species or the homologous amino acid sequence of X family polymerases of any species.

The synthesis may be performed in a microfluidic device, for example an electrowetting-on-dielectric (EWoD) device. Alternatively the synthesis may be performed in a microtitre plate format.

### BRIEF DESCRIPTION OF THE DRAWINGS

FIG. 1 shows schematically one embodiment of the invention. The cell-free protein synthesis reaction contains a nucleic acid template containing the expression cassette for the gene of interest fused to a detectable tag, which is then expressed into the protein of interest through coupled or uncoupled in-vitro transcription and in-vitro translation. The protein of interest is thus fused to a detectable peptide tag at the N- or C-termini. The nature of the detectable peptide tag is that it can be complemented with a complementary polypeptide resulting in a protein that is fluorescent.

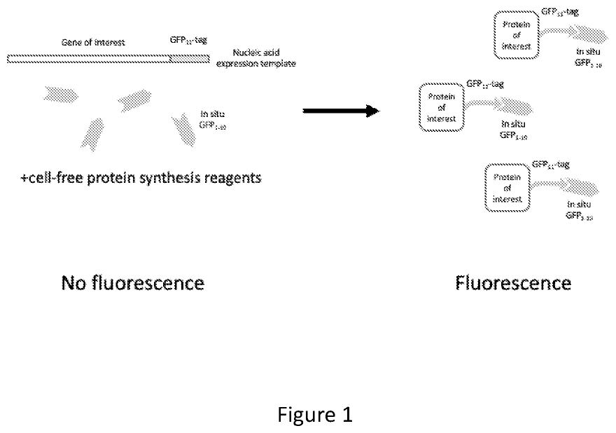

FIG. 2 PAGE gel showing ratio of protein in pellet (P) vs supernatant) (SN).

FIG. 3 Graphical representation of gel intensity ccGFP₁₋₁₀ v3—no MBP from the gel on FIG. 2. The variant sequence Cand 14 is more soluble than the wild type.

FIG. 4 Graphical representation of gel intensity ccGFP₁₋₁₀ v3 (with MBP) from the lower gel in FIG. 2. The MBP fusion further enhances solubility, and the soluble fraction is greater for the variant Cand 14.

FIG. 5 shows an absorbance spectrum for the proteins K45E plus MBP in full 1-11 and assembled 1-10 plus 11 forms. The absorbance traces are largely identical, with a max absorbance at 500 nm for each protein.

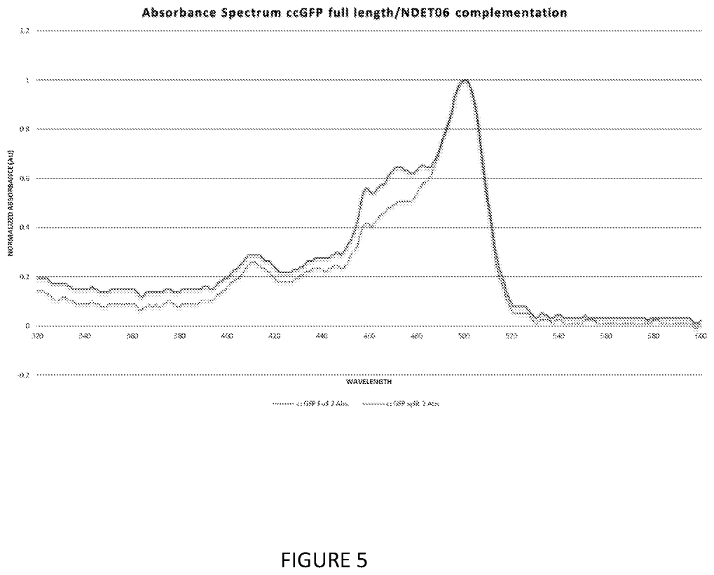

FIG. 6 shows images of fluorescent signals in droplets during protein expression using the ccGFP₁₋₁₀ variant K45E with an MBP fusion.

FIG. 7 shows complementation data for detecting an MBP recombinant protein having ccGFP₁₁. The signal from the detector scales with the amount of expressed protein. Where the detector is in excess, the signal is dependent on the amount of expression of ccGFP₁₁. The four left bars are the same as the amount of ccGFP₁₁ is limited. Where the ccGFP₁₁ is in excess, the signal is limited by the amount of detector protein. The four right bars are different and scale with the amount of ccGFP₁₁ present.

FIG. 8: shows the sequence and predicted domain structures for ccGFP₁₋₁₁ (SEQ ID NO: 79). Strand 3 covers residues GEQELKLRV (SEQ ID NO: 16). Strand 10 covers residues HKIEHRL VR (SEQ ID NO: 17).

FIG. 9: The effect of truncating the sequence of the ccGFP₁₁ binding domain on fluorescent signal (MBP-ccGFP₁₁ constructs).

FIG. 10: The effect of truncating the sequence of the ccGFP₁₁ binding domain on fluorescent signa (MBP-ccGFP₁₁ constructs). FIG. 10A shows sequences of ccGFP (SEQ ID NO: 13) and sfGFP (SEQ ID NO: 80). FIG. 10B shows the effect of truncating the sequence of ccGFP₁₁ binding domain (SEQ ID NO: 13) on fluorescent signal (MBP-ccGFP₁₁ constructs) with individual deletions and combined deletions.

FIG. 11: The experimental result from 24 different proteins expressed in a reconstituted cell-free protein synthesis system in droplets on an electrowetting on dielectric (EWoD) device. Each construct contains a GFP₁₁ tag. In the rows marked Screen, the GFP₁₋₁₀ detector species is present from the start of expression. The rows marked Endpoint shows the fluorescence signal from 10 hours expression in the absence of GFP₁₋₁₀ detector species followed by 5 hours complementation with the GFP₁₋₁₀ detector species. This experiment showed significant differences between expression/complementation in Screen BioInk compared to Endpoint detection.

### DETAILED DESCRIPTION OF THE INVENTION

Here, we report the use of the improved split peptide system for fluorescence-based monitoring of the expression of a protein of interest in cell-free protein synthesis reactions. The monitoring can be performed on device during the course of the expression, so can be used in real-time or as an end-point measurement.

Disclosed herein is a ccGFP variant comprising one or more mutations in beta strands adjacent to strand 11. Disclosed is a ccGFP variant comprising one or more mutations to beta strands 3 or 10. The amino acid position may include K45, located in strand 3. Strand 3 is amino acids 40-48, sequence GQKTLKLRV Disclosed is a ccGFP₁₋₁₁ sequence having a mutation in the strand 3 region GQKTLKLRV (residues 40-48).

Disclosed herein is a ccGFP variant comprising a K45E mutation. The variant can be a ccGFP₁₋₁₀ variant or a ccGFP₁₋₁₁ variant. The ccGFP₁₋₁₀ variant can be complexed with ccGFP₁₁ variant.

Disclosed herein is a method for the monitoring of cell-free protein synthesis in a droplet on a digital microfluidic device comprising
a. cell-free transcription and translation of a protein of interest fused to a ccGFP₁₁ peptide tag; and
b. monitoring the presence of the peptide tag using a further ccGFP₁₋₁₀ polypeptide having a K45E amino acid change, which in the presence of the ccGFP peptide tag produces a detectable signal.

Due to the increased solubility of the ccGFP₁₋₁₀, the expression of the protein of interest fused to a ccGFP₁₁ can be performed in the presence of the ccGFP₁₋₁₀. Thus monitoring of expression can be performed in real-time as the GFP₁₋₁₁ assembles. Alternatively the ccGFP₁₋₁₀ can be co-expressed.

The peptide tag can be a ccGFP₁₁ tag. The sequence of the tag can be selected from

(SEQ ID NO: 11)
GDAVQIQEHAVAKYFTV
(SEQ ID NO: 12)
GDTVQLQEHAVAKYFTV
(SEQ ID NO: 13)
GETIQLQEHAVAKYFTE

or a truncated version thereof.

The tag should contain at least the region LQEHAVAK (SEQ ID NO: 18). The tag should be at least 13 amino acids in length.

The nucleic acid sequence for expression of the tag can be a truncated version of

(SEQ ID NO: 15)
5′GGTGAAACCATCCAGTTACAAGAACACGCCGTGGCCAAATATTTCACC
GAA

The tag sequence LQEHAVAK (SEQ ID NO: 18) can be expressed using a sequence

(SEQ ID NO: 19)
5′TTACAAGAACACGCCGTGGCCAAA

Protein sequences disclosed herein may be attached to further elements to improve solubility. The variant may be attached to one or more solubility enhancing sequences. The solubility enhancing sequence may be a peptide sequence or a naturally occurring sequence. The solubility enhancing sequence may be selected from for example maltose binding protein (MBP), Small Ubiquitin-like Modifier (SUMO), Glutathione S-transferase (GST) or thioredoxin (TRX). The tags may be attached to either the C or N terminus or to both the C and N terminus. Any example of a solubility enhancer may be used. A list of possible proteins is shown below. Any sequence selected from the list below may be chosen:

| | |
|---|---|
| Glutathione S-Transferase | GST |
| Small Ubiquitin-like Modifier | SUMO |
| Maltose Binding Protein | MBP |
| Fasciola hepatica 8 kDa antigen | FH8 |
| Thioredoxin | TRX |
| Solubility Enhancing Ubiquitous Tag | SNUT |
| Seventeen kilodalton protein | SKP |
| Monomeric bacteriophage T7 orc protein | MOCR |
| E coli secreted protein A | ESPA |
| N-utilization substance | NusA |
| IgG domain B1 of Protein G | GB1 |
| IgG repeat domain ZZ of Protein A | ZZ |
| Mutated dehalogenase | HaloTag |
| Phage T7 protein kinase | T7PK |
| E. coli trypsin inhibitor | Ecotin |
| Calcium-binding protein | CaBP |
| Stress-response arsenate reductase | ArsC |
| N-terminal fragment of translation | IF2-domain 1 |
| initiation factor IF2 | |
| Stress-response protein | RpoA |
| Stress-response protein | SlyD |
| Stress-response protein | Tsf |
| Stress-response protein | RpoS |
| Stress-response protein | PotD |
| Stress-response protein | Crr |
| E. coli acidic protein | msyB |
| E. coli acidic protein | yjgD |
| E. coli acidic protein | rpoD |
| T7 phage tail | P17 |
| metal-binding protein | CUSF |
| 53-amino-acid-long N-terminal extension | NEXT |
| sequence | |

Disclosed is the use of the soluble ccGFP as a solubility enhancer during the expression process to increase the soluble yield of expressed proteins in CFPS systems. The presence of particular solubility enhancers during the expression process increases the yield of soluble expression. The protein can be expressed without the need for the synthesis of solubility factors as part of the amino acid sequence, the ccGFP₁₋₁₀ detector can be used as a solubility factors after the proteins are expressed. The attachment of solubility factors via binding tags means the expressed proteins are instantly soluble and there is no requirement for waiting for expressed sequences on the POI to correctly fold to enhance solubility. In addition the CFPS resources are not used to synthesise solubility tag proteins.

Applicants have appreciated that the presence of solubilising factors which bind to a protein rapidly after it is expressed increases the soluble yield of certain proteins. Disclosed is a method for improving the soluble yield of an expressed protein of interest (POI) in a cell or cell-free expression system by expressing the POI in the presence of a highly soluble ccGFP₁₋₁₀ moiety which binds to a binding sequence attached to the POI. The ccGFP₁₁ binding sequence may be attached to either the C or N terminus.

Disclosed is a method for the cell-free expression of peptides or proteins in a digital microfluidic device. The droplets having the components required for cell-free protein synthesis (CFPS), otherwise known as in-vitro protein synthesis, can be manipulated by electrokinesis in order to effect and improve protein expression.

Electrowetting is the modification of the wetting properties of a surface (which is typically hydrophobic) with an applied electric field. Microfluidic devices for manipulating droplets or magnetic beads based on electrowetting have been extensively described. In the case of droplets in channels this can be achieved by causing the droplets, for example in the presence of an immiscible carrier fluid, to travel through a microfluidic channel defined by the walls of a cartridge or microfluidic tubing. Embedded in the walls of the cartridge or tubing are electrodes covered with a dielectric layer each of which are connected to an A/C biasing circuit capable of being switched on and off rapidly at intervals to modify the electrowetting field characteristics of the layer. This gives rise to the ability to steer the droplet along a given path.

As an alternative to microfluidic channel systems, droplets can also be generated and manipulated on planar surfaces using digital microfluidics (DMF). In contrast to channel based microfluidics, DMF utilizes alternating currents on an electrode array for moving fluid on the surface of the array. Liquids can thus be moved on an open-plan device by electrowetting. Digital microfluidics allows precise control over the droplet movements including droplet fusion and separation.

Cell-free protein synthesis, also known as in-vitro protein synthesis or CFPS, is the production of peptides or proteins using biological machinery in a cell-free system, that is, without the use of living cells. The in-vitro protein synthesis environment is not constrained within a cell wall or limited by conditions necessary to maintain cell viability, and enables the rapid production of any desired protein from a nucleic acid template, usually plasmid DNA or RNA from an in-vitro transcription. CFPS has been known for decades, and many commercial systems are available. Cell-free protein synthesis encompasses systems based on crude lysate (Cold Spring Harb Perspect Biol. 2016 December; 8 (12): a023853) and systems based on reconstituted, purified molecular reagents, such as the PURE system for protein production (Methods Mol Biol. 2014; 1118:275-284). CFPS requires significant concentrations of biomacromolecules, including DNA, RNA, proteins, polysaccharides, molecular crowding agents, and more (Febs Letters 2013, 2, 58, 261-268).

To date, digital microfluidics, electrowetting-on-dielectric (EWoD), and electrokinesis in general have only found limited uses in cell-free biological-based applications, mostly due to biofouling, where biological components such as proteins, nucleic acids, crude cell extracts and other bioproducts adsorb and/or denature to hydrophobic surfaces. Biofouling is well known in the art to limit the ability of EWoD devices to manipulate droplets containing biomacromolecules. Wheeler and colleagues report that the maximum actuation time for droplets on EWOD devices containing biological media is 30 min before biofouling inhibits EWoD-based droplet actuation (Langmuir 2011, 27, 13, 8586-8594).

Digital microfluidics can be carried out in an air-filled system where the liquid drops are manipulated on the surface in air. However, at elevated temperatures or over prolonged periods, the volatile aqueous droplets simply dry onto the surface by evaporation. This issue is compounded by the high surface area to volume ratio of nanoliter and microliter sized drops. Hence air-filled systems are generally not suitable for protein expression where the temperature of the system needs to be maintained at a temperature suitable for enzyme activity and the duration of the synthesis needs to be prolonged for synthesized proteins levels to be detectable.

Protein expression typically requires an ample supply of oxygen. The most convenient and high yielding way to power CFPS is via oxidative phosphorylation where O₂ serves as the final electron acceptor; however, there are other ways that involve replenishing with energy molecules not involved in oxidative phosphorylation. In a confined microfluidic or digital microfluidic system of droplets, insufficient oxygen is available to enable efficient protein synthesis.

Described herein are improved methods allowing for the cell-free expression of peptides or proteins in a digital microfluidic device. Included is a method for the cell-free expression of peptides or proteins in a microfluidic device wherein the method comprises one or more droplets containing a nucleic acid template (i.e., DNA or RNA) and a cell-free system having components for protein expression in an oil-filled environment, and moving said droplets using electrokinesis. The components for the cell-free protein synthesis droplet can be pre-mixed prior to introduction to or mixed on the digital microfluidic device.

The droplet can be repeatedly moved for at least a period of 30 minutes whilst the protein is expressed. The droplet can be repeatedly moved for at least a period of two hours whilst the protein is expressed. The droplet can be repeatedly moved for at least a period of twelve hours whilst the protein is expressed. The act of moving the droplet allows oxygen to be supplied to the droplet and dispersed throughout the droplet. The act of moving improves the level of protein expression over a droplet which remains static. The droplet can be moved using any means of electrokinesis. The droplet can be moved using electrowetting-on-dielectric (EWoD). The electrical signal on the EWoD or optical EWOD device can be delivered through segmented electrodes, active-matrix thin-film transistors, or digital micromirrors. The oil in the device can be any water immiscible liquid. The oil can be mineral oil, silicone oil such as dodecamethylpentasiloxane (DMPS), an alkyl-based solvent such as decane or dodecane, or a fluorinated oil. The oil can be oxygenated prior to or during the expression process. Alternatively, the device can be an air-filled device where droplets containing cell-free protein synthesis reagents are rapidly moved into position and fixed into an array under a humidified gas to prevent evaporation. Humidification can be achieved by enclosing or sealing the digital microfluidic device and providing on-board reagent reservoirs. Additionally, humidification can be achieved by connecting an aqueous reservoir to an enclosed or sealed digital microfluidic device. The aqueous reservoir can have a defined temperature or solute concentration in order to provide specific relative humidities (e.g., a saturated potassium sulfate solution at 30° C.).

A source of supplemental oxygen can be supplied to the droplets. For example droplets or gas bubbles containing gaseous or dissolved oxygen can be merged with the droplets during the protein expression. Additionally, a source of supplemental oxygen can be found by oxygenating the oil that is used as the filler medium. It is well-known in the art that oils such as hexadecane, HFE-7500, and others can be oxygenated to support the oxygen requirements of cell growth, especially E. coli cell growth (RSC Adv., 2017, 7, 40990-40995). Oxygenation can be achieved by aerating the oil with pure oxygen or atmospheric air. Alternatively the source of oxygen can be a molecular source which releases oxygen. Alternatively the droplets can be moved to an air/liquid boundary to enable increased diffusion of oxygen from a gaseous environment. Alternatively the droplets can be presented in a humidified air filled device.

The droplets can be formed before entering the microfluidic device and flowed into the device. Alternatively the droplets can be merged on the device. Included is a method comprising merging a first droplet containing a nucleic acid template such as a plasmid with a second droplet containing a cell-free extract having the components for protein expression to form a combined droplet capable of cell-free protein synthesis.

The droplets can be split on the device either before or after expression. Included herein is a method further comprising splitting the aqueous droplet into multiple droplets. If desired the split droplets can be screened with further additives. Included is a method wherein one or more of the split droplets are merged with additive droplets for screening.

The cell-free expression of peptides or proteins can use a cell lysate having the reagents to enable protein expression. Common components of a cell-free reaction include an energy source, a supply of amino acids, cofactors such as magnesium, and the relevant enzymes. A cell extract is obtained by lysing the cell of interest and removing the cell walls, DNA genome, and other debris by centrifugation. The remains are the cell machinery including ribosomes, aminoacyl-tRNA synthetases, translation initiation and elongation factors, nucleases, etc. Once a suitable nucleic acid template is added, the nucleic acid template can be expressed as a peptide or protein using the cell derived expression machinery.

Any particular nucleic acid template can be expressed using the system described herein. Three types of nucleic acid templates used in CFPS include plasmids, linear expression templates (LETs), and mRNA. Plasmids are circular templates, which can be produced either in cells or synthetically. LETs can be made via PCR. While LETs are easier and faster to make, plasmid yields are usually higher in CFPS. mRNA can be produced through in-vitro transcription systems. The methods use a single nucleic acid template per droplet. The methods can use multiple droplets having a different nucleic acid template per droplet.

An energy source is an important part of a cell-free reaction. Usually, a separate mixture containing the needed energy source, along with a supply of amino acids, is added to the extract for the reaction. Common sources are phosphoenolpyruvate, acetyl phosphate, and creatine phosphate. The energy source can be replenished during the expression process by adding further reagents to the droplet during the process.

The cell-free extract having the components for protein expression includes everything required for protein expression apart from the nucleic acid template. Thus the term includes all the relevant ribosomes, enzymes, initiation factors, nucleotide monomers, amino acid monomers, metal ions and energy sources. Once the nucleic acid template is added, protein expression is initiated without further reagents being required.

Thus the cell-lysate can be supplemented with additional reagents prior to the template being added. The cell-free extract having the components for protein expression would typically be produced as a bulk reagent or ‘master mix’ which can be formulated into many identical droplets prior to the distinct template being separately added to separate droplets. Common cell extracts in use today are made from E. coli (ECE), rabbit reticulocytes (RRL), wheat germ (WGE), insect cells (ICE) and Yeast Kluyveromyces (the D2P system). All of these extracts are commercially available.

Rather than originating from a cell extract, the cell-free system can be assembled from the required reagents. Systems based on reconstituted, purified molecular reagents are commercially available, for example the PURE system for protein production, and can be used as supplied. The PURE system is composed of all the enzymes that are involved in transcription and translation, as well as highly purified 70S ribosomes. The protein synthesis reaction of the PURE system lacks proteases and ribonucleases, which are often present as undesired molecules in cell extracts.

The term digital microfluidic device refers to a device having a two-dimensional array of planar microelectrodes. The term excludes any devices simply having droplets in a flow of oil in a channel. The droplets are moved over the surface by electrokinetic forces by activation of particular electrodes. Upon activation of the electrodes the dielectric layer becomes less hydrophobic, thus causing the droplet to spread onto the surface. A digital microfluidic (DMF) device set-up is known in the art, and depends on the substrates used, the electrodes, the configuration of those electrodes, the use of a dielectric material, the thickness of that dielectric material, the hydrophobic layers, and the applied voltage.

Once the CFPS reagents have been enclosed in the droplets, additional reagents can be supplied by merging the original droplet with a second droplet. The second droplet can carry any desired additional reagents, including for example oxygen or ‘power’ sources, or test reagents to which it is desired to expose to the expressed protein.

The droplets can be aqueous droplets. The droplets can contain an oil immiscible organic solvent such as for example DMSO. The droplets can be a mixture of water and solvent, providing the droplets do not dissolve into the bulk oil.

The droplets can be in a bulk oil layer. A dry gaseous environment simply dries the bubbles onto the surface during the expression process, leaving comet type smears of dried material by evaporation. Thus the device is filled with liquid for the expression process. Alternatively, the aqueous droplets can be in a humidified gaseous environment. A device filled with air can be sealed and humidified in order to provide an environment that reduces evaporation of CFPS droplets.

The droplets containing the cell-free extract having the components for protein expression will therefore typically be in the oil filled environment before the nucleic acid templates are added to the droplets. The templates can be added by merging droplets on the microfluidic device. Alternatively, the templates can be added to the droplets outside the device and then flowed into the device for the expression process. For example the expression process can be initiated on the device by increasing the temperature. The expression system typically operates optimally at temperatures above standard room temperatures, for example at or above 29° C.

The expression process typically takes many hours. Thus the process should be left for at least 30 minutes or 1 hour, typically at least 2 hours. Expression can be left for at least 12 hours. During the process of expression the droplets should be moved within the device. The moving improves the process by mixing the reagents and ensuring sufficient oxygen is available within the droplet. The moving can be continuous, or can be repeated with intervening periods of non-movement.

Thus the aqueous droplet can be repeatedly moved for at least a period of 30 minutes or one hour whilst the protein is expressed. The aqueous droplet can be repeatedly moved for at least a period of two hours whilst the protein is expressed. The aqueous droplet can be repeatedly moved for at least a period of twelve hours whilst the protein is expressed. The act of moving the droplet allows mixing within the droplet, and allows oxygen or other reagents to be supplied to the droplet. The act of moving improves the level of protein expression over a droplet which remains static.

Disclosed is a method comprising transcription of a nucleic acid sequence containing a sequence of

(SEQ ID NO: 14)
5′GGTGATACCGTTCAGCTGCAAGAACATGCAGTTGCAAAATACTTTACC
GTG
(SEQ ID NO: 15)
5′GGTGAAACCATCCAGTTACAAGAACACGCCGTGGCCAAATATTTCACC
GAA

The sequence can be repeated to express multiple ccGFP₁₁ amino acid tags. The transcription can be performed in droplets on a digital microfluidics device. A fluorescent signal is obtained when the expressed protein containing the ccGFP₁₁ tag(s) complements ccGFP₁₋₁₀ protein.

Digital microfluidics (DMF) refers to a two-dimensional planar surface platform for lab-on-a-chip systems that is based upon the manipulation of microdroplets. Droplets can be dispensed, moved, stored, mixed, reacted, or analyzed on a platform with a set of insulated electrodes. Digital microfluidics can be used together with analytical analysis procedures such as mass spectrometry, colorimetry, electrochemical, and electrochemiluminescence.

The droplet can be moved using any means of electrokinesis. The aqueous droplet can be moved using electrowetting-on-dielectric (EWoD). Electrowetting on a dielectric (EWoD) is a variant of the electrowetting phenomenon that is based on dielectric materials. During EWOD, a droplet of a conducting liquid is placed on a dielectric layer with insulating and hydrophobic properties. Upon activation of the electrodes the dielectric layer becomes less hydrophobic, thus causing the droplet to spread onto the surface.

The electrical signal on the EWoD or optically-activated amorphous silicon (a-Si) EWOD device can be delivered through segmented electrodes, active-matrix thin-film transistors or digital micromirrors. Optically-activated s-Si EWOD devices are well known in the art for actuating droplets (J. Adhes. Sci. Technol., 2012, 26, 1747-1771).

The oil in the device can be any water immiscible or hydrophobic liquid. The oil can be mineral oil, silicone oil such as dodecamethylpentasiloxane (DMPS), an alkyl-based solvent such as decane or dodecane, or a fluorinated oil. The air in the device can be any humidified gas.

Through an affinity tag, such as a FLAG-tag, HIS-tag, GST-tag, MBP-tag, STREP-tag, or other form of affinity tag, CFPS-expressed proteins can be immobilized to a solid-support affinity resin and fresh batches of CFPS reagent can be delivered over the said resin. Thus, renewed reagents can be used to carry out protein synthesis, closely mimicking industrial methods of continuous flow (CF) and continuous exchange (CE) CFPS. By mimicking CF- and CE-CFPS, users can scale up their CFPS production methods.

The droplets can be actuated on a hydrophobic surface on the digital microfluidic device (ACS Nano 2018, 12, 6, 6050-6058). The hydrophobic surface can be a hydrophobic surface such as polytetrafluoroethylene (PTFE), Teflon AF (DuPont Inc), CYTOP (AGC Chemicals Inc), or FluoroPel (Cytonix LLC). The hydrophobic surface may be modified in such a way to reduce biofouling, especially biofouling resulting from exposure to CFPS reagents or nucleic acid reagents. The hydrophobic surface may also be superhydrophobic, such as NeverWet (NeverWet LLC) or Ultra-Ever Dry (Flotech Performance Systems Ltd). Superhydrophobic surfaces prevent biofouling compared with typical fluorocarbon-based hydrophobic surfaces. Superhydrophobic surfaces thus prolong the capability of digital microfluidic devices to move CFPS droplets and general solutions containing biopolymers (RSC Adv., 2017, 7, 49633-49648). The hydrophobic surface can also be a slippery liquid infused porous surface (SLIPS), which can be formed by infusing Krtox-103 oil (DuPont) with porous PTFE film (Lab Chip, 2019, 19, 2275).

Droplets can also contain additives to reduce the effects of biofouling on digital microfluidic surfaces. Specifically, droplets containing CFPS components can also contain additives such as surfactants or detergents to reduce the effects of biofouling on the hydrophobic or superhydrophobic surface of a digital microfluidic device (Langmuir 2011, 27, 13, 8586-8594). Such droplets may use antifouling additives such as TWEEN 20, Triton X-100, and/or Pluronic F127. Specifically, droplets containing CFPS components may contain TWEEN 20 at 0.1% v/v, Triton X-100 at 0.1% v/v, and/or Pluronic F127 at 0.05% w/v.

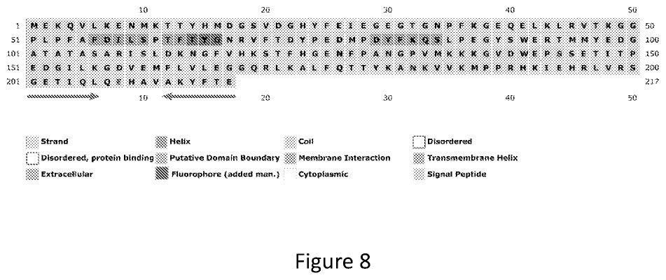

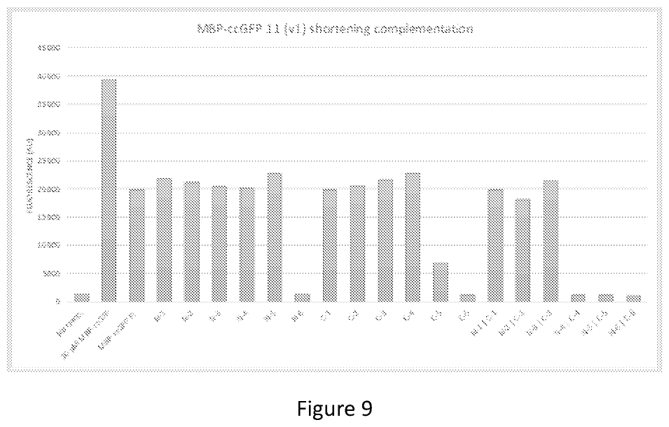

For electrowetting on dielectrics (EWoD), the change in contact angle of reagent upon the application of electric potential is an inverse function of surface tension. Thus, for low voltage EWoD operations, reduction in surface tension is achieved by addition of surfactants to reagents, which for CFPS reactions means to the lysate and to the DNA. This results in a dilution of the lysate, and it has been seen, in experiments, that diluting or otherwise adulterating the lysate results in a decrease in expression level of the protein of interest. Thus performing CFPS on DMF where the surfactants are added to the solutions being moved will necessarily result in a dilution and adulteration of the lysate and thus a decrease in the level of protein expression. In addition to being a problem in its own right, this further complicates extrapolation of on-DMF results to in-tube predictions of protein yield. An additional detriment of having to add surfactants to the samples is that this increases the time required for sample preparation, as well as increasing the potential for inconsistent results due to ‘user error,’ as there is more handling of reagents. An additional detriment of having to add surfactants to the samples is that certain downstream operations are hindered. For example, if a protein of interest is expressed in a cell-free system with a GFP₁₁ (or similar) peptide tag, it's downstream complementation with a GFP₁₋₁₀ (or similar) detector polypeptide is hindered in the presence of surfactant. This is shown in FIGS. 8 and 9. Removal of the surfactant from the aqueous phase is therefore advantageous.

Rather than adding surfactants to the aqueous sample, it is instead possible to add surfactant, such as a sorbitan ester such as Span85 (e.g. Sorbitan trioleate, Sigma Aldrich, SKU 8401240025), to the oil. This has the advantages of enabling CFPS reactions to proceed on-DMF without dilution or adulteration. Additionally, it simplifies the sample preparation procedure for setting up the reactions, increasing the ease of use and the consistency of results. Using 1% w/w Span85 in dodecane allows for dilution-free CFPS reactions on-DMF, as well as dilution-free detection of the expressed non-fluorescent proteins. Other surfactants besides Span85, and oils other than dodecane could be used. A range of concentrations of Span85 could be used. Surfactants could be nonionic, anionic, cationic, amphoteric or a mixture thereof. Oils could be mineral oils or synthetic oils, including silicone oils, petroleum oils, and perfluorinated oils. Surfactants can have a detrimental effect on (1) the CFPS reactions and (2) the efficiency of the detection system (if the detection system involves complementation of a tag and detector). For example, by performing the CFPS reaction on-DMF with oil-surfactant mix, the detection of the expressed protein can also proceed without dilution and without adding aqueous surfactant. It has been shown that surfactants reduce the efficiency of some detection systems, including but not limited to the Split ccGFP (e.g. ccGFP₁₁/ccGFP₁₋₁₀) system, so removing surfactants from the reagent mix and instead adding them to the oil can be beneficial.

The peptide tag can be attached to the C or N terminus of the protein. The protein may be fused to multiple tags. For example the protein may be fused to multiple ccGFP₁₁ peptide tags and the synthesis occurs in the presence of multiple ccGFP₁₋₁₀ polypeptides.

After expression the proteins can be purified on the device. Disclosed is a method comprising
a. taking a digital microfluidic device having a planar array of electrodes;
b. synthesising a protein of interest having a binding tag in droplets on the device;
c. capturing the proteins via the binding tags, thereby immobilising the proteins;
d. moving the droplets using the electrodes, thereby removing the synthesised proteins from the droplet;
e. optionally washing the immobilised proteins; and
f. optionally releasing the proteins into further droplets.

The binding moiety tag can be a sub-component of a fluorescent protein. Thus the fully assembled protein becomes fluorescent. For example if the immobilised material contains ccGFP₁₋₁₀ and the tag contains a ccGFP₁₁ peptide, complementation forms immobilised fluorescent ccGFP, allowing simultaneous monitoring and purification. The immobilised material can be washed and then eluted by disrupting the complemented split ccGFP, e.g. through the use of salt or temperature. Alternatively the ccGFP₁₁ tag may be removed using a protease, for example TEV or 3C protease.

The protease can be selected from the following: TEV, C3, enterokinase (EK) light chain, factor Xa (FXA), furin (FN) or thrombin. Enterokinase (EK) cleaves a NNNNL motif. Factor Xa cleaves a I(E/D)GR motif. Furin cleaves a RXXR motif. Thrombin cleaves a LVPRGS motif. TEV Protease is a cysteine protease that recognizes the sequence Glu-Asn-Leu-Tyr-Phe-Gln-(Gly/Ser) and cleaves between the Gln and Gly/Ser residues. C3 Protease is a cysteine protease that recognizes Leu-Glu-Val-Leu-Phe-Gln/Gly-Pro (LEVLFQ/GP) and cleavage occurs between the Gln and Gly-Pro residues.

Disclosed is a method comprising
a. taking a digital microfluidic device having a planar array of electrodes;
b. synthesising a protein of interest having one of more ccGFP₁₁ tags in droplets on the device;
c. capturing the proteins via the ccGFP₁₁ tags, thereby immobilising the proteins;
d. moving the droplets using the electrodes, thereby removing the synthesised proteins from the droplet;
e. optionally washing the immobilised proteins; and
f. optionally releasing the proteins into further droplets.

Immobilisation can be performed using immobilised ccGFP₁₋₁₀. Once immobilised via complementation, the proteins become fluorescent. Therefore the correctly expressed proteins can be both monitored and purified by complexing with the immobilised ccGFP₁₋₁₀.

Affinity tags may be appended to proteins so that they can be purified from their crude biological source using an affinity technique. The purification tags may be selected from for example FLAG-tag, His-tag, GST-tag, MBP-tag, STREP-tag. The Flag® tag, also known as the DYKDDDDK-tag, is a popular protein tag that is commonly used in affinity chromatography and protein research. His tags are polyhistidine strings of amino acids, typically between 6 and 9 histidine amino acids in length.

The binding moiety for purification may contain four or more amino acids. The binding sequences may contain 4-30 amino acids. The binding moiety may be selected from:

Alfa-tag
(SEQ ID NO: 20)
(SRLEEELRRRLTE)
Avi-tag
(SEQ ID NO: 21)
(GLNDIFEAQKIEWHE)
C-tag
(SEQ ID NO: 22)
(EPEA)
Calmodulin-tag
(SEQ ID NO: 23)
(KRRWKKNFIAVSAANRFKKISSSGAL)
Dogtag
(SEQ ID NO: 24)
(DIPATYEFTDGKHYITNEPIPPK)
E-tag
(SEQ ID NO: 25)
(GAPVPYPDPLEPR)
FLAG
(SEQ ID NO: 26)
(DYKDDDDK)
G4T
(SEQ ID NO: 27)
(EELLSKNYHLENEVARLKK)
HA
(SEQ ID NO: 28)
(YPYDVPDYA)
His
(SEQ ID NO: 29)
(HHHHHH)
Isopeptag
(SEQ ID NO: 30)
(TDKDMTITFTNKKDAE)
lanthanide binding tag (LBT)
(SEQ ID NO: 31)
(FIDTNNDGWIEGDELLLEEG)
Myc
(SEQ ID NO: 32)
(EQKLISEEDL)
NE-Tag
(SEQ ID NO: 33)
(TKENPRSNQEESYDDNES)
Poly Glutamate-tag
(SEQ ID NO: 34)
(EEEEEEE)
Poly Arginine-tag
(SEQ ID NO: 35)
(RRRRRRR)
Rho1D4-tag
(SEQ ID NO: 36)
(TETSQVAPA)
SBP-tag
(SEQ ID NO: 37)
(MDEKTTGWRGGHVVEGLAGELEQLRARLEHHPQGQREP)
Sdytag
(SEQ ID NO: 38)
(DPIVMIDNDKPIT)
SH3
(SEQ ID NO: 39)
(STVPVAPPRRRRG)
SNAC
(SEQ ID NO: 40)
(GSHHW)
Snooptag
(SEQ ID NO: 41)
(KLGDIEFIKVNK)
Softag 1
(SEQ ID NO: 42)
(SLAELLNAGLGGS)
Softag 3
(SEQ ID NO: 43)
(TQDPSRVG)
Spot-tag
(SEQ ID NO: 44)
(PDRVRAVSHWSS)
Spytag
(SEQ ID NO: 45)
(AHIVMVDAYKPTK)
S-tag
(SEQ ID NO: 46)
(KETAAAKFERQHMDS)
Strep-tag
(SEQ ID NO: 47)
(AWAHPQPGG)
(SEQ ID NO: 48)
(AWRHPQFGG)
Strep-tag II
(SEQ ID NO: 49)
(WSHPQFEK)
T7tag
(SEQ ID NO: 50)
(MASMTGGQQMG)
TC-tag
(SEQ ID NO: 51)
(EVHTNQDPLD)
Ty-tag
(SEQ ID NO: 52)
(CCPGCC)
VSV-tag
(SEQ ID NO: 53)
(YTDIEMNRLGK)
Xpress-tag
(SEQ ID NO: 54)
(DLYDDDDK)

After expression, proteins may be purified, for example by binding to particles such as magnetic beads,

The ccGFP₁₋₁₀ species may be attached to purification tags to enable purification of the expressed protein. The ccGFP₁₋₁₀ species may be attached to solubility tags to further enhance solubility of the expressed protein. Disclosed herein is a ccGFP₁₋₁₀ species having one or more mutations in strand 3 attached to an affinity purification tag and a solubility tag. The solubility tag may be selected from:

| | |
|---|---|
| Glutathione S-Transferase | GST |
| Small Ubiquitin-like Modifier | SUMO |
| Maltose Binding Protein | MBP |
| Fasciola hepatica 8 kDa antigen | FH8 |
| Thioredoxin | TRX |
| Solubility Enhancing Ubiquitous Tag | SNUT |
| Seventeen kilodalton protein | SKP |
| Monomeric bacteriophage T7 orc protein | MOCR |
| E coli secreted protein A | ESPA |
| N-utilization substance | NusA |
| IgG domain B1 of Protein G | GB1 |
| IgG repeat domain ZZ of Protein A | ZZ |
| Mutated dehalogenase | HaloTag |
| Phage T7 protein kinase | T7PK |
| E. coli trypsin inhibitor | Ecotin |
| Calcium-binding protein | CaBP |
| Stress-response arsenate reductase | ArsC |
| N-terminal fragment of translation | IF2-domain 1 |
| initiation factor IF2 | |
| Stress-response protein | RpoA |
| Stress-response protein | SlyD |
| Stress-response protein | Tsf |
| Stress-response protein | RpoS |
| Stress-response protein | PotD |
| Stress-response protein | Crr |
| E. coli acidic protein | msyB |
| E. coli acidic protein | yjgD |
| E. coli acidic protein | rpoD |
| T7 phage tail | P17 |
| metal-binding protein | CUSF |
| 53-amino-acid-long N-terminal extension | NEXT |
| sequence | |

The purification tag may be selected from:

Alfa-tag
(SEQ ID NO: 20)
(SRLEEELRRRLTE)
Avi-tag
(SEQ ID NO: 21)
(GLNDIFEAQKIEWHE)
C-tag
(SEQ ID NO: 22)
(EPEA)
Calmodulin-tag
(SEQ ID NO: 23)
(KRRWKKNFIAVSAANRFKKISSSGAL)
Dogtag
(SEQ ID NO: 24)
(DIPATYEFTDGKHYITNEPIPPK)
E-tag
(SEQ ID NO: 25)
(GAPVPYPDPLEPR)
FLAG
(SEQ ID NO: 26)
(DYKDDDDK)
G4T
(SEQ ID NO: 27)
(EELLSKNYHLENEVARLKK)
HA
(SEQ ID NO: 28)
(YPYDVPDYA)
His
(SEQ ID NO: 29)
(HHHHHH)
Isopeptag
(SEQ ID NO: 30)
(TDKDMTITFTNKKDAE)
lanthanide binding tag (LBT)
(SEQ ID NO: 31)
(FIDTNNDGWIEGDELLLEEG)
Myc
(SEQ ID NO: 32)
(EQKLISEEDL)
NE-Tag
(SEQ ID NO: 33)
(TKENPRSNQEESYDDNES)
Poly Glutamate-tag
(SEQ ID NO: 34)
(EEEEEEE)
Poly Arginine-tag
(SEQ ID NO: 35)
(RRRRRRR)
Rho1D4-tag
(SEQ ID NO: 36)
(TETSQVAPA)
SBP-tag
(SEQ ID NO: 37)
(MDEKTTGWRGGHVVEGLAGELEQLRARLEHHPQGQREP)
Sdytag
(SEQ ID NO: 38)
(DPIVMIDNDKPIT)
SH3
(SEQ ID NO: 39)
(STVPVAPPRRRRG)
SNAC
(SEQ ID NO: 40)
(GSHHW)
Snooptag
(SEQ ID NO: 41)
(KLGDIEFIKVNK)
Softag 1
(SEQ ID NO: 42)
(SLAELLNAGLGGS)
Softag 3
(SEQ ID NO: 43)
(TQDPSRVG)
Spot-tag
(SEQ ID NO: 44)
(PDRVRAVSHWSS)
Spytag
(SEQ ID NO: 45)
(AHIVMVDAYKPTK)
S-tag
(SEQ ID NO: 46)
(KETAAAKFERQHMDS)
Strep-tag
(SEQ ID NO: 47)
(AWAHPQPGG)
(SEQ ID NO: 48)
(AWRHPQFGG)
Strep-tag II
(SEQ ID NO: 49)
(WSHPQFEK)
T7tag
(SEQ ID NO: 50)
(MASMTGGQQMG)
TC-tag
(SEQ ID NO: 51)
(EVHTNQDPLD)
Ty-tag
(SEQ ID NO: 52)
(CCPGCC)
VSV-tag
(SEQ ID NO: 53)
(YTDIEMNRLGK)
Xpress-tag
(SEQ ID NO: 54)
(DLYDDDDK)

Disclosed herein is a ccGFP₁₋₁₀ species having one or more mutations in strand 3 attached to an affinity poly His purification tag. Disclosed herein is a ccGFP₁₋₁₀ species having one or more mutations in strand 3 attached to a P17 solubility tag. Disclosed herein is a ccGFP₁₋₁₀ species having one or more mutations in strand 3 attached to a NEXT solubility tag. Disclosed herein is a ccGFP₁₋₁₀ species having one or more mutations in strand 3 attached to a Fh8 solubility tag. Disclosed herein is a ccGFP₁₋₁₀ species having one or more mutations in strand 3 attached to a Trx solubility tag. Disclosed herein is a ccGFP₁₋₁₀ species having one or more mutations in strand 3 attached to a Mocr solubility tag. Disclosed herein is a ccGFP₁₋₁₀ species having one or more mutations in strand 3 attached to a SNUT solubility tag. Disclosed herein is a ccGFP₁₋₁₀ species having one or more mutations in strand 3 attached to a GST solubility tag. Disclosed herein is a ccGFP₁₋₁₀ species having one or more mutations in strand 3 attached to a CusF solubility tag. Disclosed herein is a ccGFP₁₋₁₀ species having one or more mutations in strand 3 attached to an MBP solubility tag. Disclosed herein is a ccGFP₁₋₁₀ species having one or more mutations in strand 3 attached to a ZZ solubility tag. Disclosed herein is a ccGFP₁₋₁₀ species having one or more mutations in strand 3 attached to a poly His purification tag and a solubility tag. In each case the ccGFP species can have a modification at K45, such as K45E.

Disclosed herein are detector species which binds to ccGFP₁₁ comprising the sequence:

P17
(SEQ ID NO: 55)
MSMEKQVLKENMKTTYHMDGSVDGHYFEIEGEGTGNPFKGEQELELRVTKGGPLPFAFDIL
SPTFTYGNRVFTDYPEDMPDYFKQSLPEGYSWERTMMYEDGATATASARISLDKNGFVHKS
TFHGENFPANGPVMKKKGVDWEPSSETITPEDGILKGDVEMFLVLEGGQRLKALFQTTYKA
NKVVKMPPRHKIEHRLVRSGSGAEAAAKEAAAKAGSGMKNESSTNATNTKQWRDETKGFR
DEAKRFKNTAGGGGGSHHHHHH.
NEXT
(SEQ ID NO: 56)
MSMEKQVLKENMKTTYHMDGSVDGHYFEIEGEGTGNPFKGEQELELRVTKGGPLPFAFDIL
SPTFTYGNRVFTDYPEDMPDYFKQSLPEGYSWERTMMYEDGATATASARISLDKNGFVHKS
TFHGENFPANGPVMKKKGVDWEPSSETITPEDGILKGDVEMFLVLEGGQRLKALFQTTYKA
NKVVKMPPRHKIEHRLVRSGSGAEAAAKEAAAKAGSGMAVQHSNAPLIDLGAEMKKQHKE
AAPEGAAPAQGKAPAAEAKKEEAPKPKPVVGGGGSHHHHHH.
Fh8
(SEQ ID NO: 57)
MSMEKQVLKENMKTTYHMDGSVDGHYFEIEGEGTGNPFKGEQELELRVTKGGPLPFAFDIL
SPTFTYGNRVFTDYPEDMPDYFKQSLPEGYSWERTMMYEDGATATASARISLDKNGFVHKS
TFHGENFPANGPVMKKKGVDWEPSSETITPEDGILKGDVEMFLVLEGGQRLKALFQTTYKA
NKVVKMPPRHKIEHRLVRSGSGAEAAAKEAAAKAGSGMPSVQEVEKLLHVLDRNGDGKVS
AEELKAFADDSKCPLDSNKIKAFIKEHDKNKDGKLDLKELVSILSSGGGGSHHHHHH.
Trx
(SEQ ID NO: 58)
MSMEKQVLKENMKTTYHMDGSVDGHYFEIEGEGTGNPFKGEQELELRVTKGGPLPFAFDIL
SPTFTYGNRVFTDYPEDMPDYFKQSLPEGYSWERTMMYEDGATATASARISLDKNGFVHKS
TFHGENFPANGPVMKKKGVDWEPSSETITPEDGILKGDVEMFLVLEGGQRLKALFQTTYKA
NKVVKMPPRHKIEHRLVRSGSGAEAAAKEAAAKAGSGMSDKIIHLTDDSFDTDVLKADGAI
LVDFWAEWCGPCKMIAPILDEIADEYQGKLTVAKLNIDQNPGTAPKYGIRGIPTLLLFKNGEV
AATKVGALSKGQLKEFLDANLAGGGGSHHHHHH.
Mocr
(SEQ ID NO: 59)
MSMEKQVLKENMKTTYHMDGSVDGHYFEIEGEGTGNPFKGEQELELRVTKGGPLPFAFDIL
SPTFTYGNRVFTDYPEDMPDYFKQSLPEGYSWERTMMYEDGATATASARISLDKNGFVHKS
TFHGENFPANGPVMKKKGVDWEPSSETITPEDGILKGDVEMFLVLEGGQRLKALFQTTYKA
NKVVKMPPRHKIEHRLVRSGSGAEAAAKEAAAKAGSGMAMSNMTYNNVFDHAYEMLKEN
IRYDDIRDTDDLHDAIHMAADNAVPHYYADIFSVMASEGIDLEFEDSGLMPDTKDVIRILQAR
IYEQLTIDLWEDAEDLLNEYLEEVEEYEEDEEGGGGSHHHHHH.
SNUT
(SEQ ID NO: 60)
MSMEKQVLKENMKTTYHMDGSVDGHYFEIEGEGTGNPFKGEQELELRVTKGGPLPFAFDIL
SPTFTYGNRVFTDYPEDMPDYFKQSLPEGYSWERTMMYEDGATATASARISLDKNGFVHKS
TFHGENFPANGPVMKKKGVDWEPSSETITPEDGILKGDVEMFLVLEGGQRLKALFQTTYKA
NKVVKMPPRHKIEHRLVRSGSGAEAAAKEAAAKAGSGMKPHIDNYLHDKDKDERIEQYDK
NVKEQASKDKKQQAKPQIPKDKSKVAGYIEIPDADIKEPVYPGPATPEQLNRGVSFAEENESL
DDQNISIAGHTFIDRPNYQFTNLKAAKKGSMVYFKVGNETRKYKMTSIRDVKPTDVEVLDGS
GGGGSHHHHHH.
GST
(SEQ ID NO: 61)
MSMEKQVLKENMKTTYHMDGSVDGHYFEIEGEGTGNPFKGEQELELRVTKGGPLPFAFDIL
SPTFTYGNRVFTDYPEDMPDYFKQSLPEGYSWERTMMYEDGATATASARISLDKNGFVHKS
TFHGENFPANGPVMKKKGVDWEPSSETITPEDGILKGDVEMFLVLEGGQRLKALFQTTYKA
NKVVKMPPRHKIEHRLVRSGSGAEAAAKEAAAKAGSGMSPILGYWKIKGLVQPTRLLLEYL
EEKYEEHLYERDEGDKWRNKKFELGLEFPNLPYYIDGDVKLTQSMAIIRYIADKHNMLGGCP
KERAEISMLEGAVLDIRYGVSRIAYSKDFETLKVDFLSKLPEMLKMFEDRLCHKTYLNGDHV
THPDFMLYDALDVVLYMDPMCLDAFPKLVCFKKRIEAIPQIDKYLKSSKYIAWPLQGWQAT
FGGGDHPPKGGGGSHHHHHH.
MBP
(SEQ ID NO: 62)
MSMEKQVLKENMKTTYHMDGSVDGHYFEIEGEGTGNPFKGEQELELRVTKGGPLPFAFDIL
SPTFTYGNRVFTDYPEDMPDYFKQSLPEGYSWERTMMYEDGATATASARISLDKNGFVHKS
TFHGENFPANGPVMKKKGVDWEPSSETITPEDGILKGDVEMFLVLEGGQRLKALFQTTYKA
NKVVKMPPRHKIEHRLVRSGSGAEAAAKEAAAKAGSGMKIEEGKLVIWINGDKGYNGLAE
VGKKFEKDTGIKVTVEHPDKLEEKFPQVAATGDGPDIIFWAHDRFGGYAQSGLLAEITPDKA
FQDKLYPFTWDAVRYNGKLIAYPIAVEALSLIYNKDLLPNPPKTWEEIPALDKELKAKGKSAL
MFNLQEPYFTWPLIAADGGYAFKYENGKYDIKDVGVDNAGAKAGLTFLVDLIKNKHMNAD
TDYSIAEAAFNKGETAMTINGPWAWSNIDTSKVNYGVTVLPTFKGQPSKPFVGVLSAGINAA
SPNKELAKEFLENYLLTDEGLEAVNKDKPLGAVALKSYEEELAKDPRIAATMENAQKGEIMP
NIPQMSAFWYAVRTAVINAASGRQTVDEALKDAQTGGGGSHHHHHH.
CusF
(SEQ ID NO: 63)
MSMEKQVLKENMKTTYHMDGSVDGHYFEIEGEGTGNPFKGEQELELRVTKGGPLPFAFDIL
SPTFTYGNRVFTDYPEDMPDYFKQSLPEGYSWERTMMYEDGATATASARISLDKNGFVHKS
TFHGENFPANGPVMKKKGVDWEPSSETITPEDGILKGDVEMFLVLEGGQRLKALFQTTYKA
NKVVKMPPRHKIEHRLVRSGSGAEAAAKEAAAKAGSGMANEHHHETMSEAQPQVISATGV
VKGIDLESKKITIHHDPIAAVNWPEMTMRFTITPQTKMSEIKTGDKVAFNFVQQGNLSLLQDI
KVSQGGGGSHHHHHH.
ZZ
(SEQ ID NO: 64)
MSMEKQVLKENMKTTYHMDGSVDGHYFEIEGEGTGNPFKGEQELELRVTKGGPLPFAFDIL
SPTFTYGNRVFTDYPEDMPDYFKQSLPEGYSWERTMMYEDGATATASARISLDKNGFVHKS
TFHGENFPANGPVMKKKGVDWEPSSETITPEDGILKGDVEMFLVLEGGQRLKALFQTTYKA
NKVVKMPPRHKIEHRLVRSGSGAEAAAKEAAAKAGSGMVDNKFNKEQQNAYYEILHLPNL
NEGQRNAFIQSLKDDPSQSANLLAEAKKLNDAQAPKVDNKFNKEQQNAFYEILHLPNLNEEQ
RNAFIQSLKDDPSQSANLLAEAEKLNDAQAPKGGGGSHHHHHH.

Disclosed herein are nucleic acid sequences for expressing said detector species comprising the nucleic acid sequences:

MBP
(SEQ ID NO: 65)
ATGAGCATGGAAAAACAGGTGCTGAAAGAAAACATGAAAACCACCTATCACATGGATG
GTAGCGTTGATGGTCACTATTTTGAAATTGAAGGTGAAGGCACCGGCAATCCGTTTAAAG
GTGAACAAGAACTGGAACTCCGTGTTACCAAAGGTGGTCCGCTGCCGTTTGCATTTGATA
TTCTGAGCCCGACCTTTACCTATGGTAATCGTGTTTTTACCGACTATCCGGAAGATATGCC
GGATTATTTCAAACAGAGCCTGCCGGAAGGTTATAGCTGGGAACGTACCATGATGTATG
AAGATGGTGCAACCGCAACCGCCAGCGCACGTATTAGCCTGGATAAAAATGGTTTTGTG
CATAAGAGCACCTTTCACGGTGAAAACTTTCCGGCAAATGGTCCGGTTATGAAAAAGAA
AGGTGTTGATTGGGAACCGAGCAGCGAAACCATTACACCGGAAGATGGTATTCTGAAAG
GTGATGTTGAAATGTTTCTGGTTCTGGAAGGTGGTCAGCGTCTGAAAGCCCTGTTTCAGA
CCACCTATAAAGCCAATAAAGTGGTTAAAATGCCTCCGCGTCATAAAATTGAACATCGTC
TGGTTCGTAGCGGTAGTGGTGCAGAAGCTGCTGCAAAAGAGGCAGCTGCGAAGGCAGGT
AGTGGTATGAAAATCGAAGAAGGTAAACTGGTAATCTGGATTAACGGCGATAAAGGCTA
TAACGGTCTCGCTGAAGTCGGTAAGAAATTCGAGAAAGATACCGGAATTAAAGTCACCG
TTGAGCATCCGGATAAACTGGAAGAGAAATTCCCACAGGTTGCGGCAACTGGCGATGGC
CCTGACATTATCTTCTGGGCACACGACCGCTTTGGTGGCTACGCTCAATCTGGCCTGTTG
GCTGAAATCACCCCGGACAAAGCGTTCCAGGACAAGCTGTATCCGTTTACCTGGGATGCC
GTACGTTACAACGGCAAGCTGATTGCTTACCCGATCGCTGTTGAAGCGTTATCGCTGATT
TATAACAAAGATCTGCTGCCGAACCCGCCAAAAACCTGGGAAGAGATCCCGGCGCTGGA
TAAAGAACTGAAAGCGAAAGGTAAGAGCGCGCTGATGTTCAACCTGCAAGAACCGTACT
TCACCTGGCCGCTGATTGCTGCTGACGGGGGTTATGCGTTCAAGTATGAAAACGGCAAGT
ACGACATTAAAGACGTGGGCGTGGATAACGCTGGCGCGAAAGCGGGTCTGACCTTCCTG
GTTGACCTGATTAAAAACAAACACATGAATGCAGACACCGATTACTCCATCGCAGAAGC
TGCCTTTAATAAAGGCGAAACAGCGATGACCATCAACGGCCCGTGGGCATGGTCCAACA
TCGACACCAGCAAAGTGAATTATGGTGTAACGGTACTGCCGACCTTCAAGGGTCAACCA
TCCAAACCGTTCGTTGGCGTGCTGAGCGCAGGTATTAACGCCGCCAGTCCGAACAAAGA
GCTGGCGAAAGAGTTCCTCGAAAACTATCTGCTGACTGATGAAGGTCTGGAAGCGGTTA
ATAAAGACAAACCGCTGGGTGCCGTAGCGCTGAAGTCTTACGAGGAAGAGTTGGCGAAA
GATCCACGTATTGCCGCCACCATGGAAAACGCCCAGAAAGGTGAAATCATGCCGAACAT
C
CCGCAGATGTCCGCTTTCTGGTATGCCGTGCGTACTGCGGTGATCAACGCCGCCAGCGGT
CGTCAGACTGTCGATGAAGCCCTGAAAGACGCGCAGACTGGCGGTGGCGGTAGCCATCA
CCACCATCATCACTAA.
P17
(SEQ ID NO: 66)
ATGAGCATGGAAAAACAGGTGCTGAAAGAAAACATGAAAACCACCTATCACATGGATG
GTAGCGTTGATGGTCACTATTTTGAAATTGAAGGTGAAGGCACCGGCAATCCGTTTAAAG
GTGAACAAGAACTGGAACTCCGTGTTACCAAAGGTGGTCCGCTGCCGTTTGCATTTGATA
TTCTGAGCCCGACCTTTACCTATGGTAATCGTGTTTTTACCGACTATCCGGAAGATATGCC
GGATTATTTCAAACAGAGCCTGCCGGAAGGTTATAGCTGGGAACGTACCATGATGTATG
AAGATGGTGCAACCGCAACCGCCAGCGCACGTATTAGCCTGGATAAAAATGGTTTTGTG
CATAAGAGCACCTTTCACGGTGAAAACTTTCCGGCAAATGGTCCGGTTATGAAAAAGAA
AGGTGTTGATTGGGAACCGAGCAGCGAAACCATTACACCGGAAGATGGTATTCTGAAAG
GTGATGTTGAAATGTTTCTGGTTCTGGAAGGTGGTCAGCGTCTGAAAGCCCTGTTTCAGA
CCACCTATAAAGCCAATAAAGTGGTTAAAATGCCTCCGCGTCATAAAATTGAACATCGTC
TGGTTCGTAGCGGTAGTGGTGCAGAAGCTGCTGCAAAAGAGGCAGCTGCGAAGGCAGGT
AGTGGTATGAAGAACGAGAGCAGCACAAATGCGACAAATACCAAGCAGTGGCGCGACG
AGACCAAGGGTTTCCGCGACGAGGCAAAACGTTTCAAAAACACTGCGGGAGGCGGTGGC
GGTAGCCATCACCACCATCATCACTAA.
NEXT
(SEQ ID NO: 67)
ATGAGCATGGAAAAACAGGTGCTGAAAGAAAACATGAAAACCACCTATCACATGGATG
GTAGCGTTGATGGTCACTATTTTGAAATTGAAGGTGAAGGCACCGGCAATCCGTTTAAAG
GTGAACAAGAACTGGAACTCCGTGTTACCAAAGGTGGTCCGCTGCCGTTTGCATTTGATA
TTCTGAGCCCGACCTTTACCTATGGTAATCGTGTTTTTACCGACTATCCGGAAGATATGCC
GGATTATTTCAAACAGAGCCTGCCGGAAGGTTATAGCTGGGAACGTACCATGATGTATG
AAGATGGTGCAACCGCAACCGCCAGCGCACGTATTAGCCTGGATAAAAATGGTTTTGTG
CATAAGAGCACCTTTCACGGTGAAAACTTTCCGGCAAATGGTCCGGTTATGAAAAAGAA
AGGTGTTGATTGGGAACCGAGCAGCGAAACCATTACACCGGAAGATGGTATTCTGAAAG
GTGATGTTGAAATGTTTCTGGTTCTGGAAGGTGGTCAGCGTCTGAAAGCCCTGTTTCAGA
CCACCTATAAAGCCAATAAAGTGGTTAAAATGCCTCCGCGTCATAAAATTGAACATCGTC
TGGTTCGTAGCGGTAGTGGTGCAGAAGCTGCTGCAAAAGAGGCAGCTGCGAAGGCAGGT
AGTGGTATGGCCGTACAGCATAGTAACGCTCCTTTAATCGACCTGGGTGCAGAAATGAA
GAAACAACATAAAGAGGCCGCGCCAGAGGGTGCCGCGCCTGCACAAGGAAAGGCCCCA
GCAGCAGAGGCAAAAAAAGAGGAAGCTCCAAAACCCAAACCGGTCGTGGGCGGTGGCG
GTAGCCATCACCACCATCATCACTAA.
Fh8
(SEQ ID NO: 68)
ATGAGCATGGAAAAACAGGTGCTGAAAGAAAACATGAAAACCACCTATCACATGGATG
GTAGCGTTGATGGTCACTATTTTGAAATTGAAGGTGAAGGCACCGGCAATCCGTTTAAAG
GTGAACAAGAACTGGAACTCCGTGTTACCAAAGGTGGTCCGCTGCCGTTTGCATTTGATA
TTCTGAGCCCGACCTTTACCTATGGTAATCGTGTTTTTACCGACTATCCGGAAGATATGCC
GGATTATTTCAAACAGAGCCTGCCGGAAGGTTATAGCTGGGAACGTACCATGATGTATG
AAGATGGTGCAACCGCAACCGCCAGCGCACGTATTAGCCTGGATAAAAATGGTTTTGTG
CATAAGAGCACCTTTCACGGTGAAAACTTTCCGGCAAATGGTCCGGTTATGAAAAAGAA
AGGTGTTGATTGGGAACCGAGCAGCGAAACCATTACACCGGAAGATGGTATTCTGAAAG
GTGATGTTGAAATGTTTCTGGTTCTGGAAGGTGGTCAGCGTCTGAAAGCCCTGTTTCAGA
CCACCTATAAAGCCAATAAAGTGGTTAAAATGCCTCCGCGTCATAAAATTGAACATCGTC
TGGTTCGTAGCGGTAGTGGTGCAGAAGCTGCTGCAAAAGAGGCAGCTGCGAAGGCAGGT
AGTGGTATGCCCAGTGTACAAGAAGTAGAAAAACTATTACATGTTCTAGATAGGAATGG
AGACGGCAAGGTGTCTGCCGAAGAATTAAAAGCATTTGCTGACGATTCCAAATGTCCTTT
GGACTCAAATAAAATTAAAGCTTTTATAAAAGAACATGATAAAAATAAGGATGGTAAAC
TTGATTTAAAAGAGCTTGTAAGTATTTTGTCATCTGGCGGTGGCGGTAGCCATCACCACC
ATCATCACTAA.
MBP
(SEQ ID NO: 69)
ATGAGCATGGAAAAACaggtgctgaaagaaaacatgaaaaccacctatcacatggatggtagcgttgatggtcactattttgaaattg
aaggtgaaggcaccggcaatccgtttaaaggtgaacaagaactgGaactcCgtgttaccaaaggtggtccgctgccgtttgcatttgatattctga
gcccgacctttacctatggtaatcgtgtttttaccgactatccggaagatatgccggattatttcaaacagagcctgccggaaggttatagctgggaac
gtaccatgatgtatgaagatggtgcaaccgcaaccgccagcgcacgtattagcctggataaaaatggttttgtgcataagagcacctttcacggtga
aaactttccggcaaatggtccggttatgaaaaagaaaggtgttgattgggaaccgagcagcgaaaccattacaccggaagatggtattctgaaagg
tgatgttgaaatgtttctggttctggaaggtggtcagcgtctgaaagccctgtttcagaccacctataaagccaataaagtggttaaaatgcctccgcgt
cataaaattgaacatcgtctggttcgtagcGGTAGTGGTGCAGAAGCTGCTGCAAAAGAGGCAGCTGCGAA
GGCAGGTAGTGGTATGAAAATCGAAGAAGGTAAACTGGTAATCTGGATTAACGGCGATA
AAGGCTATAACGGTCTCGCTGAAGTCGGTAAGAAATTCGAGAAAGATACCGGAATTAAA
GTCACCGTTGAGCATCCGGATAAACTGGAAGAGAAATTCCCACAGGTTGCGGCAACTGG
CGATGGCCCTGACATTATCTTCTGGGCACACGACCGCTTTGGTGGCTACGCTCAATCTGG
CCTGTTGGCTGAAATCACCCCGGACAAAGCGTTCCAGGACAAGCTGTATCCGTTTACCTG
GGATGCCGTACGTTACAACGGCAAGCTGATTGCTTACCCGATCGCTGTTGAAGCGTTATC
GCTGATTTATAACAAAGATCTGCTGCCGAACCCGCCAAAAACCTGGGAAGAGATCCCGG
CGCTGGATAAAGAACTGAAAGCGAAAGGTAAGAGCGCGCTGATGTTCAACCTGCAAGAA
CCGTACTTCACCTGGCCGCTGATTGCTGCTGACGGGGGTTATGCGTTCAAGTATGAAAAC
GGCAAGTACGACATTAAAGACGTGGGCGTGGATAACGCTGGCGCGAAAGCGGGTCTGAC
CTTCCTGGTTGACCTGATTAAAAACAAACACATGAATGCAGACACCGATTACTCCATCGC
AGAAGCTGCCTTTAATAAAGGCGAAACAGCGATGACCATCAACGGCCCGTGGGCATGGT
CCAACATCGACACCAGCAAAGTGAATTATGGTGTAACGGTACTGCCGACCTTCAAGGGT
CAACCATCCAAACCGTTCGTTGGCGTGCTGAGCGCAGGTATTAACGCCGCCAGTCCGAAC
AAAGAGCTGGCGAAAGAGTTCCTCGAAAACTATCTGCTGACTGATGAAGGTCTGGAAGC
GGTTAATAAAGACAAACCGCTGGGTGCCGTAGCGCTGAAGTCTTACGAGGAAGAGTTGG
CGAAAGATCCACGTATTGCCGCCACCATGGAAAACGCCCAGAAAGGTGAAATCATGCCG
AACATCCCGCAGATGTCCGCTTTCTGGTATGCCGTGCGTACTGCGGTGATCAACGCCGCC
AGCGGTCGTCAGACTGTCGATGAAGCCCTGAAAGACGCGCAGACTGGCGGTGGCGGTAG
CCATCACCACCATCATCACTAA
TRX
(SEQ ID NO: 70)
ATGAGCATGGAAAAACAGGTGCTGAAAGAAAACATGAAAACCACCTATCACATGGATG
GTAGCGTTGATGGTCACTATTTTGAAATTGAAGGTGAAGGCACCGGCAATCCGTTTAAAG
GTGAACAAGAACTGGAACTCCGTGTTACCAAAGGTGGTCCGCTGCCGTTTGCATTTGATA
TTCTGAGCCCGACCTTTACCTATGGTAATCGTGTTTTTACCGACTATCCGGAAGATATGCC
GGATTATTTCAAACAGAGCCTGCCGGAAGGTTATAGCTGGGAACGTACCATGATGTATG
AAGATGGTGCAACCGCAACCGCCAGCGCACGTATTAGCCTGGATAAAAATGGTTTTGTG
CATAAGAGCACCTTTCACGGTGAAAACTTTCCGGCAAATGGTCCGGTTATGAAAAAGAA
AGGTGTTGATTGGGAACCGAGCAGCGAAACCATTACACCGGAAGATGGTATTCTGAAAG
GTGATGTTGAAATGTTTCTGGTTCTGGAAGGTGGTCAGCGTCTGAAAGCCCTGTTTCAGA
CCACCTATAAAGCCAATAAAGTGGTTAAAATGCCTCCGCGTCATAAAATTGAACATCGTC
TGGTTCGTAGCGGTAGTGGTGCAGAAGCTGCTGCAAAAGAGGCAGCTGCGAAGGCAGGT
AGTGGTATGTCAGATAAAATAATTCATTTAACAGATGATAGTTTTGATACTGATGTATTG
AAAGCAGATGGAGCTATCCTCGTTGATTTTTGGGCTGAATGGTGTGGACCCTGTAAAATG
ATTGCACCTATTTTAGATGAAATTGCTGATGAATATCAAGGTAAATTAACAGTCGCTAAA
TTAAATATTGATCAAAATCCAGGTACTGCTCCAAAATATGGAATTAGAGGAATACCTACT
CTTTTATTATTTAAAAATGGCGAAGTGGCTGCAACAAAAGTGGGAGCTTTATCTAAAGGT
CAACTAAAAGAATTTTTAGATGCAAATCTTGCAGGCGGTGGCGGTAGCCATCACCACCAT
CATCACTAA.
MOCR
(SEQ ID NO: 71)
ATGAGCATGGAAAAACAGGTGCTGAAAGAAAACATGAAAACCACCTATCACATGGATG
GTAGCGTTGATGGTCACTATTTTGAAATTGAAGGTGAAGGCACCGGCAATCCGTTTAAAG
GTGAACAAGAACTGGAACTCCGTGTTACCAAAGGTGGTCCGCTGCCGTTTGCATTTGATA
TTCTGAGCCCGACCTTTACCTATGGTAATCGTGTTTTTACCGACTATCCGGAAGATATGCC
GGATTATTTCAAACAGAGCCTGCCGGAAGGTTATAGCTGGGAACGTACCATGATGTATG
AAGATGGTGCAACCGCAACCGCCAGCGCACGTATTAGCCTGGATAAAAATGGTTTTGTG
CATAAGAGCACCTTTCACGGTGAAAACTTTCCGGCAAATGGTCCGGTTATGAAAAAGAA
AGGTGTTGATTGGGAACCGAGCAGCGAAACCATTACACCGGAAGATGGTATTCTGAAAG
GTGATGTTGAAATGTTTCTGGTTCTGGAAGGTGGTCAGCGTCTGAAAGCCCTGTTTCAGA
CCACCTATAAAGCCAATAAAGTGGTTAAAATGCCTCCGCGTCATAAAATTGAACATCGTC
TGGTTCGTAGCGGTAGTGGTGCAGAAGCTGCTGCAAAAGAGGCAGCTGCGAAGGCAGGT
AGTGGTATGGCAATGTCTAATATGACCTATAATAATGTTTTTGATCATGCATATGAGATG
TTAAAAGAAAACATAAGATATGATGATATTAGAGACACAGATGATCTTCATGATGCTATT
CATATGGCAGCAGATAACGCAGTTCCTCATTATTATGCTGATATTTTTAGTGTCATGGCAT
CAGAAGGCATTGATCTAGAGTTTGAAGATTCTGGGCTTATGCCAGATACGAAAGACGTA
ATAAGGATATTACAAGCAAGAATTTACGAACAATTAACTATAGATTTATGGGAAGATGC
TGAAGACTTGTTAAATGAATATTTAGAAGAAGTGGAGGAATACGAAGAAGATGAAGAA
GGCGGTGGCGGTAGCCATCACCACCATCATCACTAA.
SNUT
(SEQ ID NO: 72)
ATGAGCATGGAAAAACAGGTGCTGAAAGAAAACATGAAAACCACCTATCACATGGATG
GTAGCGTTGATGGTCACTATTTTGAAATTGAAGGTGAAGGCACCGGCAATCCGTTTAAAG
GTGAACAAGAACTGGAACTCCGTGTTACCAAAGGTGGTCCGCTGCCGTTTGCATTTGATA
TTCTGAGCCCGACCTTTACCTATGGTAATCGTGTTTTTACCGACTATCCGGAAGATATGCC
GGATTATTTCAAACAGAGCCTGCCGGAAGGTTATAGCTGGGAACGTACCATGATGTATG
AAGATGGTGCAACCGCAACCGCCAGCGCACGTATTAGCCTGGATAAAAATGGTTTTGTG
CATAAGAGCACCTTTCACGGTGAAAACTTTCCGGCAAATGGTCCGGTTATGAAAAAGAA
AGGTGTTGATTGGGAACCGAGCAGCGAAACCATTACACCGGAAGATGGTATTCTGAAAG
GTGATGTTGAAATGTTTCTGGTTCTGGAAGGTGGTCAGCGTCTGAAAGCCCTGTTTCAGA
CCACCTATAAAGCCAATAAAGTGGTTAAAATGCCTCCGCGTCATAAAATTGAACATCGTC
TGGTTCGTAGCGGTAGTGGTGCAGAAGCTGCTGCAAAAGAGGCAGCTGCGAAGGCAGGT
AGTGGTATGAAGCCTCACATTGACAATTATTTACACGACAAGGACAAAGACGAGCGCAT
TGAGCAATACGACAAAAATGTCAAGGAACAAGCAAGCAAGGACAAGAAACAGCAGGCG
AAGCCTCAAATCCCCAAGGACAAAAGTAAAGTCGCTGGGTACATCGAGATCCCGGATGC
AGACATTAAGGAGCCCGTCTATCCTGGACCTGCAACACCTGAGCAGCTTAATCGCGGGG
TGTCGTTTGCGGAAGAAAATGAGTCGTTGGACGACCAGAATATCAGCATCGCAGGCCAC
ACCTTTATCGACCGCCCCAATTATCAGTTCACAAACTTGAAAGCGGCCAAGAAGGGTTCA
ATGGTATATTTCAAGGTTGGAAATGAGACGCGCAAATACAAGATGACAAGTATCCGTGA
CGTCAAACCTACTGACGTAGAAGTACTTGATGGTTCCGGCGGTGGCGGTAGCCATCACCA
CCATCATCACTAA.
GST
(SEQ ID NO: 73)
ATGAGCATGGAAAAACAGGTGCTGAAAGAAAACATGAAAACCACCTATCACATGGATG
GTAGCGTTGATGGTCACTATTTTGAAATTGAAGGTGAAGGCACCGGCAATCCGTTTAAAG
GTGAACAAGAACTGGAACTCCGTGTTACCAAAGGTGGTCCGCTGCCGTTTGCATTTGATA
TTCTGAGCCCGACCTTTACCTATGGTAATCGTGTTTTTACCGACTATCCGGAAGATATGCC
GGATTATTTCAAACAGAGCCTGCCGGAAGGTTATAGCTGGGAACGTACCATGATGTATG
AAGATGGTGCAACCGCAACCGCCAGCGCACGTATTAGCCTGGATAAAAATGGTTTTGTG
CATAAGAGCACCTTTCACGGTGAAAACTTTCCGGCAAATGGTCCGGTTATGAAAAAGAA
AGGTGTTGATTGGGAACCGAGCAGCGAAACCATTACACCGGAAGATGGTATTCTGAAAG
GTGATGTTGAAATGTTTCTGGTTCTGGAAGGTGGTCAGCGTCTGAAAGCCCTGTTTCAGA
CCACCTATAAAGCCAATAAAGTGGTTAAAATGCCTCCGCGTCATAAAATTGAACATCGTC
TGGTTCGTAGCGGTAGTGGTGCAGAAGCTGCTGCAAAAGAGGCAGCTGCGAAGGCAGGT
AGTGGTATGTCCCCAATACTAGGTTATTGGAAAATTAAAGGCCTTGTTCAACCCACTCGA
CTATTATTGGAATATCTTGAAGAAAAATATGAAGAACATTTGTATGAACGCGATGAAGG
TGATAAATGGAGAAATAAAAAATTTGAATTGGGATTGGAATTTCCAAATTTACCTTATTA
TATTGATGGTGATGTAAAATTAACACAATCTATGGCAATAATTCGTTATATAGCTGACAA
GCACAACATGTTGGGTGGTTGTCCAAAAGAAAGAGCAGAAATTTCAATGTTAGAAGGAG
CGGTTCTAGATATTAGATACGGTGTTTCTAGAATTGCATATAGTAAAGACTTTGAAACTT
TAAAAGTTGATTTTCTTAGCAAGTTGCCTGAAATGCTGAAAATGTTCGAAGATAGGTTAT
GTCATAAAACATATTTAAATGGTGATCACGTAACCCATCCTGACTTTATGTTATATGATG
CTCTTGATGTTGTTTTATATATGGACCCAATGTGTCTGGATGCTTTTCCAAAATTAGTTTG
TTTTAAAAAAAGGATTGAAGCTATTCCTCAAATAGATAAATATTTAAAAAGCAGTAAAT
ATATAGCTTGGCCACTTCAGGGATGGCAAGCAACCTTTGGTGGTGGAGATCATCCTCCAA
AA GGCGGTGGCGGTAGCCATCACCACCATCATCACTAA.
CusF
(SEQ ID NO: 74)
ATGAGCATGGAAAAACAGGTGCTGAAAGAAAACATGAAAACCACCTATCACATGGATG
GTAGCGTTGATGGTCACTATTTTGAAATTGAAGGTGAAGGCACCGGCAATCCGTTTAAAG
GTGAACAAGAACTGGAACTCCGTGTTACCAAAGGTGGTCCGCTGCCGTTTGCATTTGATA
TTCTGAGCCCGACCTTTACCTATGGTAATCGTGTTTTTACCGACTATCCGGAAGATATGCC
GGATTATTTCAAACAGAGCCTGCCGGAAGGTTATAGCTGGGAACGTACCATGATGTATG
AAGATGGTGCAACCGCAACCGCCAGCGCACGTATTAGCCTGGATAAAAATGGTTTTGTG
CATAAGAGCACCTTTCACGGTGAAAACTTTCCGGCAAATGGTCCGGTTATGAAAAAGAA
AGGTGTTGATTGGGAACCGAGCAGCGAAACCATTACACCGGAAGATGGTATTCTGAAAG
GTGATGTTGAAATGTTTCTGGTTCTGGAAGGTGGTCAGCGTCTGAAAGCCCTGTTTCAGA
CCACCTATAAAGCCAATAAAGTGGTTAAAATGCCTCCGCGTCATAAAATTGAACATCGTC
TGGTTCGTAGCGGTAGTGGTGCAGAAGCTGCTGCAAAAGAGGCAGCTGCGAAGGCAGGT
AGTGGTATGGCTAACGAACATCATCATGAAACCATGAGCGAAGCACAACCACAGGTTAT
TAGCGCCACTGGCGTGGTAAAGGGTATCGATCTGGAAAGCAAAAAAATCACCATCCATC
ACGATCCGATTGCTGCCGTGAACTGGCCGGAGATGACCATGCGCTTTACCATCACCCCGC
AGACGAAAATGAGTGAAATTAAAACCGGCGACAAAGTGGCGTTTAATTTTGTCCAGCAG
GGCAACCTTTCTTTATTACAGGATATTAAAGTCAGCCAGGGCGGTGGCGGTAGCCATCAC
CACCATCATCACTAA.
ZZ
(SEQ ID NO: 75)
ATGAGCATGGAAAAACAGGTGCTGAAAGAAAACATGAAAACCACCTATCACATGGATG
GTAGCGTTGATGGTCACTATTTTGAAATTGAAGGTGAAGGCACCGGCAATCCGTTTAAAG
GTGAACAAGAACTGGAACTCCGTGTTACCAAAGGTGGTCCGCTGCCGTTTGCATTTGATA
TTCTGAGCCCGACCTTTACCTATGGTAATCGTGTTTTTACCGACTATCCGGAAGATATGCC
GGATTATTTCAAACAGAGCCTGCCGGAAGGTTATAGCTGGGAACGTACCATGATGTATG
AAGATGGTGCAACCGCAACCGCCAGCGCACGTATTAGCCTGGATAAAAATGGTTTTGTG
CATAAGAGCACCTTTCACGGTGAAAACTTTCCGGCAAATGGTCCGGTTATGAAAAAGAA
AGGTGTTGATTGGGAACCGAGCAGCGAAACCATTACACCGGAAGATGGTATTCTGAAAG
GTGATGTTGAAATGTTTCTGGTTCTGGAAGGTGGTCAGCGTCTGAAAGCCCTGTTTCAGA
CCACCTATAAAGCCAATAAAGTGGTTAAAATGCCTCCGCGTCATAAAATTGAACATCGTC
TGGTTCGTAGCGGTAGTGGTGCAGAAGCTGCTGCAAAAGAGGCAGCTGCGAAGGCAGGT
AGTGGTATGGTTGATAACAAATTCAATAAAGAACAGCAAAACGCATATTACGAGATTCT
TCATCTGCCGAATTTGAATGAAGGCCAACGTAATGCGTTTATCCAGTCCCTTAAAGACGA
TCCTTCCCAGTCTGCGAACTTGTTAGCGGAGGCCAAAAAATTAAACGATGCCCAAGCTCC
CAAGGTGGATAATAAGTTCAATAAGGAACAACAGAATGCTTTTTACGAAATCTTGCACCT
GCCCAATCTTAACGAAGAACAACGCAATGCTTTCATTCAAAGTCTGAAAGACGATCCCTC
GCAAAGTGCGAACTTATTGGCCGAGGCTGAGAAACTTAATGACGCTCAAGCGCCCAAGG
GCGGTGGCGGTAGCCATCACCACCATCATCACTAA.

### Devices

The manipulation of droplets by the application of electrical potential can be achieved on electrodes covered with an insulator or a dielectric or a series of insulators or dielectrics. Droplet manipulation as a result of an applied electrical potential is known as electrowetting. Electrokinesis occurs as result of a non-uniform electric field that influences the hydrostatic equilibrium of a dielectric liquid (dielectrophoresis or DEP) or a change in the contact angle of the liquid on solid surface (electrowetting-on-dielectric or EWoD). DEP can also be used to create forces on polarizable particles to induce their movement. The electrical signal can be transmitted to a discrete electrode, a transistor, an array of transistors, or a sheet of semi-conductor film whose electrical properties can be modulated by an optical signal.

EWoD phenomena occur when droplets are actuated between two parallel electrodes covered with a hydrophobic insulator or dielectric. The electric field at the electrode-electrolyte interface induces a change in the surface tension, which results in droplet motion as a result of a change in droplet contact angle. The electrowetting effect can be quantitatively treated using Young-Lippmann equation:

$$cos θ - cos {θ}_{0} = ( 1 / 2 γ LG ) c . {V}^{2}$$

where θ₀ is the contact angle when the electric field across the interfacial layer is zero, γLG is the liquid-gas tension, c is the specific capacitance (given as εᵣ. ε₀/t, where εᵣ is dielectric constant of the insulator/dielectric, ε₀ is permittivity of vacuum, t is thickness) and V is the applied voltage or electrical potential. The change in contact angle (inducing droplet movement) is thus a function of surface tension, electrical potential, dielectric thickness, and dielectric constant.

When a droplet is actuated by EWOD, there are two opposing sets of forces that act upon it: an electrowetting force induced by electric field and resistant forces that include the drag forces resulting from the interaction of the droplet with filler medium and the contact line friction (ref). The minimum voltage applied to balance the electrowetting force with the sum of all drag forces (threshold voltage) is variably determined by the thickness-to-dielectric contact ratio of the insulator/dielectric, (t/εᵣ)^{1/2}. Thus, to reduce actuation voltage, it is required to reduce (t/εᵣ)^{1/2} (i.e., increase dielectric constant or decrease insulator/dielectric thickness). To achieve low voltage actuation, thin insulator/dielectric layers must be used. However, the deposition of high quality thin insulator/dielectric layers is a technical challenge, and these thin layers are easily damaged before the desired electrowetting contact angle is large enough to drive the droplet is achieved. Most academic studies thus report the use of much higher voltages >100V on easily fabricated, thick dielectric films (>3 μm) to effect electrowetting.

High voltage EWoD-based devices with thick dielectric films, however, have limited industrial applicability largely due to their limited droplet multiplexing capability. The use of low voltage devices including thin-film transistors (TFT) and optically-activated amorphous silicon layers (a-Si) have paved the way for the industrial adoption of EWoD-based devices due to their greater flexibility in addressing electrical signals in a highly multiplex fashion. The driving voltage for TFTs or optically-activated a-Si are low (typically <15 V). The bottleneck for fabrication and thus adoption of low voltage devices has been the technical challenge of depositing high quality, thin film insulators/dielectrics. Hence there has been a particular need for improving the fabrication and composition of thin film insulator/dielectric devices.

Typically, the electrodes (or the array elements) used for EWOD are covered with (i) a hydrophilic insulator/dielectric and a hydrophobic coating or (ii) a hydrophobic insulator/dielectric. Commonly used hydrophobic coatings comprise of fluoropolymers such as Teflon AF 1600 or CYTOP. The thickness of this material as a hydrophobic coating on the dielectric is typically <100 nm and can have defects in the form of pinholes or a porous structure; hence, it is particularly important that the insulator/dielectric is pinhole free to avoid electrical shorting. Teflon has also been used as an insulator/dielectric, but it has higher voltage requirements due to its low dielectric constant and the thickness required to make it pinhole free. Other hydrophobic insulator/dielectric materials can include polymer-based dielectrics such as those based on siloxane, epoxy (e.g. SU-8), or parylene (e.g., parylene N, parylene C, parylene D, or parylene HT). Due to minimal contact angle hysteresis and a higher contact angle with aqueous solutions, Teflon is still used as a hydrophobic topcoat on these insulator/dielectric polymers. However, there are difficulties in reliably producing <1 micron pinhole-free coatings of parylene or SU-8; thus, the thickness of these materials is typically kept at a 2-5 microns at the cost of increased voltage requirements for electrowetting. It has also been reported that traditional EWoD devices with parylene C are easily broken and unstable for repeated droplet manipulation with cell culture medium. Multi-layer insulator devices deposited with metal-oxide and parylene C films have been used to produce a more robust insulator/dielectric and enable operations with lower applied voltages. Inorganic materials, such metal oxides and semiconductor oxides, commonly used in the CMOS industry as “gate dielectrics”, have been used as insulator/dielectric for EWOD devices. They offer the advantage of utilizing standard cleanroom processes for thin film depositions (<100 nm). These materials are inherently hydrophilic, requiring an additional hydrophobic coating, and can be prone to pinhole formation as a result of thin film layer deposition process. Together with the need for lower voltage operations of EWOD, recent developmental work has focused on (1) using materials with improved dielectric properties (e.g., using high-dielectric constant insulators/dielectrics), (2) optimizing the fabrication process to make the insulator/dielectric pinhole free to avoid dielectric breakdown.

Operation of EWOD devices suffers from contact angle saturation and hysteresis, which is believed to be brought about by either one or combination of these phenomena: (1) entrapment of charges in the hydrophobic film or insulator/dielectric interface, (2) adsorption of ions, (3) thermodynamic contact angle instabilities, (4) dielectric breakdown of dielectric layer, (5) the electrode-electrode-insulator interface capacitance (arising from the double layer effect), and (6) fouling of the surface (such as by biomacromolecules). One of the adverse effects of this hysteresis is reduced operational lifetime of the EWoD-based device.

Contact angle hysteresis is believed to be a result of charge accumulation at the interface or within the hydrophobic insulator after several operations. The required actuation voltage increases due to this charging phenomenon resulting in eventual catastrophic dielectric breakdown. The most probable explanation is that pinholes at the insulator/dielectric may allow the liquid to come into contact with the electrode causing electrolysis. Electrolysis is further facilitated by pinhole-prone or porous hydrophobic insulators.

Most of the studies to understand contact angle hysteresis on EWoD have been conducted on short time scales and with low conductivity solutions. Long duration actuations (e.g., >1 hour) and high conductivity solutions (e.g., 1 M NaCl) could produce several effects other than electrolysis. The ions in solution can permeate through the hydrophobic coat (under the applied electric field) and interact with the underlying insulator/dielectric. Ion permeation can result in (1) change in dielectric constant due to charge entrapment (which is different from interfacial charging) and (2) change in surface potential of a pH sensitive metal oxide. Both can result in reduction of electrowetting forces to manipulate aqueous droplets, leading to contact angle hysteresis. The inventors have previously found that the damage from high conductivity solutions reduces or disables electrowetting on electrodes by inhibiting the modulation of contact angle when an electric field is applied.

An electrokinetic device includes a first substrate having a matrix of electrodes, wherein each of the matrix electrodes is coupled to a thin film transistor, and wherein the matrix electrodes are overcoated with a functional coating comprising: a dielectric layer in contact with the matrix electrodes, a conformal layer in contact with the dielectric layer, and a hydrophobic layer in contact with the conformal layer; a second substrate comprising a top electrode; a spacer disposed between the first substrate and the second substrate and defining an electrokinetic workspace; and a voltage source operatively coupled to the matrix electrodes.

The dielectric layer may comprise silicon dioxide, silicon oxynitride, silicon nitride, hafnium oxide, yttrium oxide, lanthanum oxide, titanium dioxide, aluminum oxide, tantalum oxide, hafnium silicate, zirconium oxide, zirconium silicate, barium titanate, lead zirconate titanate, strontium titanate, or barium strontium titanate. The dielectric layer may be between 10 nm and 100 μm thick. Combinations of more than one material may be used, and the dielectric layer may comprise more than one sublayer that may be of different materials.

The conformal layer may comprise a parylene, a siloxane, or an epoxy. It may be a thin protective parylene coating in between the insulating dielectric and the hydrophobic coating. Typically, parylene is used as a dielectric layer on simple devices. In this invention, the rationale for deposition of parylene is not to improve insulation/dielectric properties such as reduction in pinholes, but rather to act as a conformal layer between the dielectric and hydrophobic layers. The inventors find that parylene, as opposed to other similar insulating coatings of the same thickness such as PDMS (polydimethylsiloxane), prevent contact angle hysteresis caused by high conductivity solutions or solutions deviating from neutral pH for extended hours. The conformal layer may be between 10 nm and 100 μm thick.

The hydrophobic layer may comprise a fluoropolymer coating, fluorinated silane coating, manganese oxide polystyrene nanocomposite, zinc oxide polystyrene nanocomposite, precipitated calcium carbonate, carbon nanotube structure, silica nanocoating, or slippery liquid-infused porous coating.

The elements may comprise one or more of a plurality of array elements, each element containing an element circuit; discrete electrodes; a thin film semiconductor in which the electrical properties can be modulated by incident light; and a thin film photoconductor whose properties can be modulated by incident light.

The functional coating may include a dielectric layer comprising silicon nitride, a conformal layer comprising parylene, and a hydrophobic layer comprising an amorphous fluoropolymer. This has been found to be a particularly advantageous combination.

The electrokinetic device may include a controller to regulate a voltage provided to the individual matrix electrodes. The electrokinetic device may include a plurality of scan lines and a plurality of gate lines, wherein each of the thin film transistors is coupled to a scan line and a gate line, and the plurality of gate lines are operatively connected to the controller. This allows all the individual elements to be individually controlled.

The second substrate may also comprise a second hydrophobic layer disposed on the second electrode. The first and second substrates may be disposed so that the hydrophobic layer and the second hydrophobic layer face each other, thereby defining the electrokinetic workspace between the hydrophobic layers.

The method is particularly suitable for aqueous droplets with a volume of 1 μL or smaller.

The EWoD-based devices shown and described below are active matrix thin film transistor devices containing a thin film dielectric coating with a Teflon hydrophobic top coat. These devices are based on devices described in the E Ink Corp patent filing on “Digital microfluidic devices including dual substrate with thin-film transistors and capacitive sensing”, US patent application no 2019/0111433, incorporated herein by reference.

Described herein are electrokinetic devices, including:
a first substrate having a matrix of electrodes, wherein each of the matrix electrodes is coupled to a thin film transistor, and wherein the matrix electrodes are overcoated with a functional coating comprising:
a dielectric layer in contact with the matrix electrodes,
a conformal layer in contact with the dielectric layer, and
a hydrophobic layer in contact with the conformal layer;
   a second substrate comprising a top electrode;
   a spacer disposed between the first substrate and the second substrate and defining an electrokinetic workspace; and
   a voltage source operatively coupled to the matrix electrodes;

Described herein is an electrokinetic device, including:
a first substrate having a matrix of electrodes, wherein each of the matrix electrodes is coupled to a thin film transistor, and wherein the matrix electrodes are overcoated with a functional coating comprising:
one or more dielectric layer(s) comprising silicon nitride, hafnium oxide or aluminum oxide in contact with the matrix electrodes,
a conformal layer comprising parylene in contact with the dielectric layer, and
a hydrophobic layer in contact with the conformal layer;
   a second substrate comprising a top electrode;
   a spacer disposed between the first substrate and the second substrate and defining an electrokinetic workspace; and
   a voltage source operatively coupled to the matrix electrodes;

The electrokinetic devices as described may be used with other elements, such as for example devices for heating and cooling the device or reagent cartridges for the introduction of reagents as needed.

### EXAMPLES

### Preparation and Analysis of Amino Acid Variants

Starter cultures of ccGFP₁₋₁₀ v3 K45E and ccGFP₁₋₁₀ v3 (WT) plus and minus MBP were generated. Colonies were chosen and incubated overnight at 37° C. The material was lysed and centrifuged at 21000 rcf for 10 min at 20° C. The supernatant (SN) having soluble protein was removed. The pellet (P) was resuspended with a needle and syringe and transferred to a fresh tune (insoluble protein). Protein gels were performed to measure soluble (SN) and insoluble fractions (P):
Mix 5 μL of soluble and insoluble sample each, mix with 5 μL of protein loading buffer+DTT
Samples were heated to 95° C. for 5 min
Transfer 4 μL of each sample to a PAGE
PAGE was performed for 20 min at 250 V
The gel was stained for 20-30 min in Coomassie

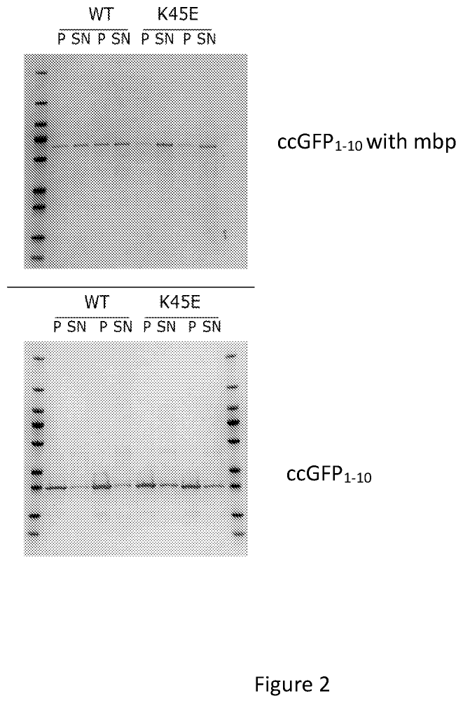

The sample bands of the PAGE of the four general conditions were quantified with GelAnalyzer. Supernatant and Pellet of each sample were added up to a 100% and the relative ratio was then calculated. The gel image is shown in FIG. 2

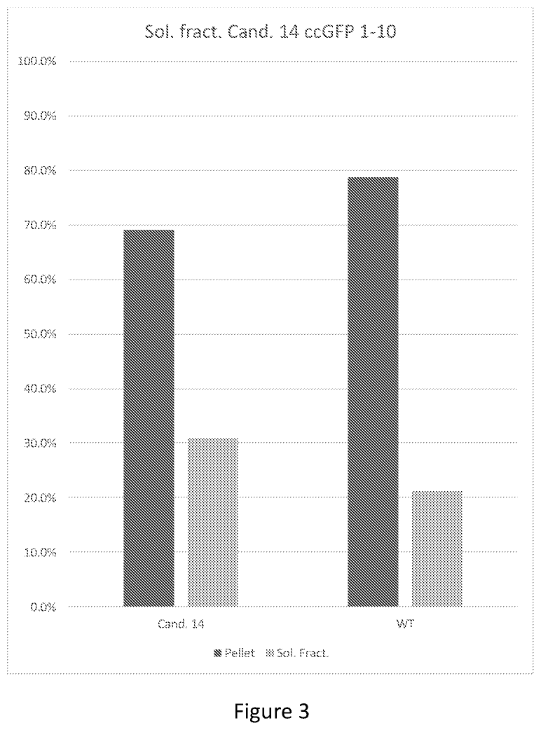

ccGFP₁₋₁₀ v3 (no MBP) (FIG. 3)

| | Pellet | Sol. Fract. | Stdv pellet | Stdv sol. |
|---|---|---|---|---|
| Cand. 14 | 69.1% | 30.9% | 0.26% | 1.64% |
| WT | 78.8% | 21.2% | 22.85% | 2.80% |

| | Sol. increase |
|---|---|
| Cand. 14 | 45.6% |

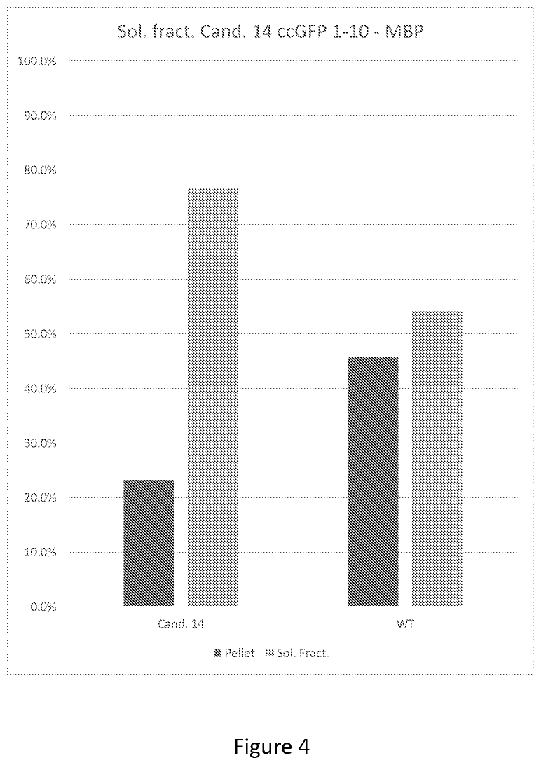

ccGFP₁₋₁₀ v3 - MBP (FIG. 4)

| | Pellet | Sol. Fract. | Stdv pellet | Stdv sol. |
|---|---|---|---|---|
| Cand. 14 | 23.2% | 76.8% | 2.10% | 16.66% |
| WT | 45.9% | 54.1% | 9.59% | 9.66% |

| | Sol. increase |
|---|---|
| Cand. 14 | 41.8% |

The ccGFP₁₋₁₀ v3 K45E variant is more soluble than the v3 “WT”. The C-Terminal MBP fusion protein with a stiff alpha-helical linker is also more soluble, in general, than the free protein alone. The soluble fraction of ccGFP₁₋₁₀ v3 K45E was 30-40%, while the MBP fusion protein of the variant was 60-75%, with an advantage of the detergent fraction. The change of K45 to E45 gave a roughly 40% increase in solubility. The MBP fusion ccGFP₁₋₁₀ v3 K45E (NDET06) has been prepared up to 130 mg/mL without any signs of precipitation.

### Use of Variants for CFPS on an Electrowetting Device.

### Methods

To analyse the variant MBP-K45E ccGFP₁₋₁₀ (NDET06) detection properties, an electrowetting on device apparatus (eDrop) experiment was set up to express proteins having a ccGFP₁₁ tag.

A mixture of different nucleic acid templates were expressed in four different protein expression systems in order to test levels of expression and detection. The NDET06 is present in the expression buffer. A comparable experiment with unmodified ccGFP₁₋₁₀ is not possible as the detection protein is insufficiently soluble even as the MBP conjugate.

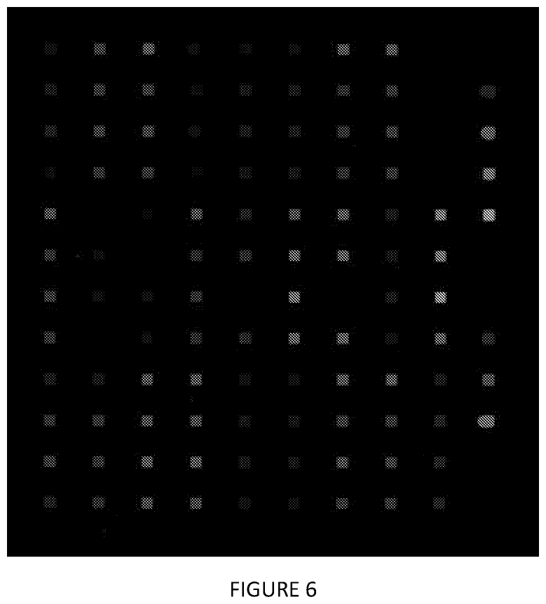

Results can be seen in FIG. 6. Each droplet contains the same amount of NDET06. The difference in fluorescence measures the varying amounts of expression of proteins carrying the ccGFP₁₁ tag. The NDET06 detector can therefore act a real-time measure of protein expression as well as an end point measure.

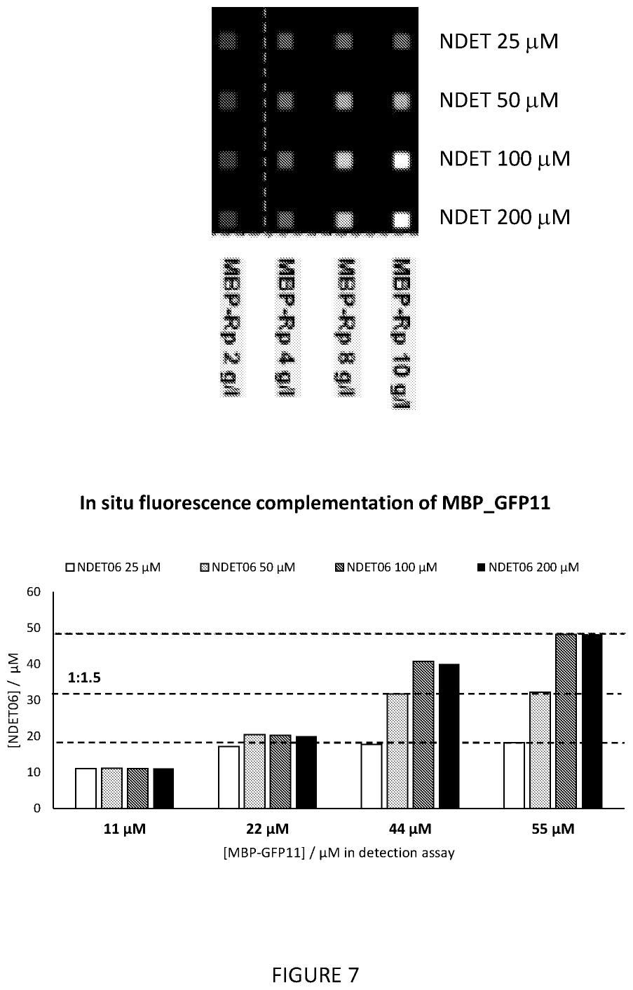

To analyse the optimal NDET06 concentration in the expression system, a further experiment was set up with four known concentrations of recombinant MBP-ccGFP₁₁. The amount of detector was varied. The results of the fluorescence intensity from the droplets is shown in FIG. 7. The signal from the detector scales with the amount of expressed protein. Where the detector is in excess, the signal is dependent on the amount of expression of ccGFP₁₁. The four left bars are the same as the amount of ccGFP₁₁ is limited. Where the ccGFP₁₁ is in excess, the signal is limited by the amount of detector protein. The four right bars are different and scale with the amount of ccGFP₁₁ present.

### Summary of Findings

NDET06 can detect soluble proteins with a molar excess of 1.5 times the ccGFP₁₁ tagged proteins. The NDET is highly soluble and can be used directly in the expression systems at concentrations of up to at least 200 μM. The optimal concentration detected when NDET06 is added in lysate is 50 μM. Considering the optimal NDET06 concentrations established, the detector is able to quantify at least up to 30 μM of soluble proteins.

### CONCLUSIONS

Adding variant K45E with an MBP fusion (NDET06) in the expression system is possible to:
Reduce the time to results as NDET06 detects soluble proteins from the beginning of expression;
Achieve higher sensitivity as the CPFS reaction and therefore the fluorescent signal is not diluted by the addition of NDET06;
Ease loading the device as NDET06 and expression systems are loaded as one reagent;
Allow more space on the device as NDET06 doesn't occupy space on the device as a separate reagent.
   Truncations of the ccGFP₁₁ Tag.

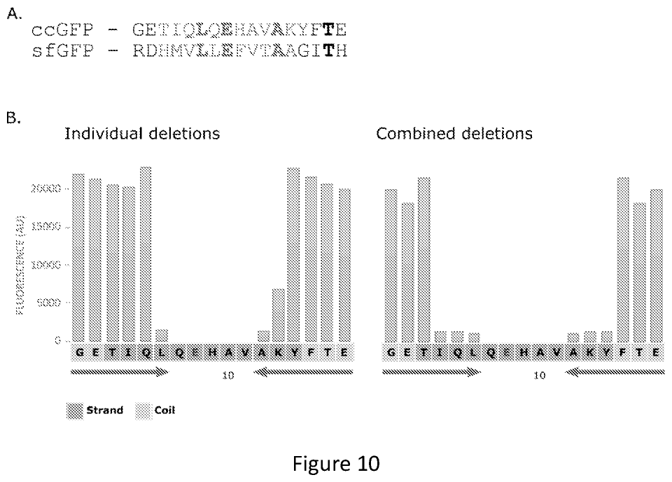

The purified DNA of the full length and 18 shortened MBP-ccGFP₁₁ (v1) variants were used in a reconstituted in vitro transcription/translation reaction together with a negative (−DNA) control and a positive (ccGFP₁₁ (v1) purified protein) control. Aliquots of the samples were analyzed and relative MBP-ccGFP₁₁ (v1) expression quantified by PAGE before complementation with ccGFP₁₋₁₀. FIGS. 9 and 10 show the effect of shortening the ccGFP₁₁ GETIQLQEHA VAKYFTE tag (SEQ ID NO: 13). The region GETIQ can be removed, leaving LQEHAVAKYFTE (SEQ ID NO: 76). The region YFTE can be removed, leaving GETIQLQEHAVAK (SEQ ID NO: 77). Up to three amino acids can be removed from each end, leaving IQLQEHAVAKY (SEQ ID NO: 78).

Expression on EWOD Device with End Point Detection

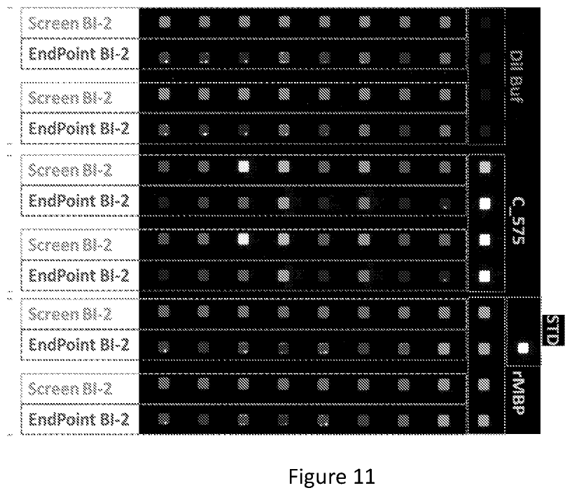

FIG. 11 shows the experimental result from 24 different proteins expressed in a reconstituted cell-free protein synthesis system in droplets on an electrowetting on dielectric (EWoD) device. Each construct contains a GFP₁₁ tag. In the rows marked Screen, the GFP₁₋₁₀ detector species is present from the start of expression. The rows marked Endpoint shows the fluorescence signal from 10 hours expression in the absence of GFP₁₋₁₀ detector species followed by 5 hours complementation with the GFP₁₋₁₀ detector species. This experiment showed significant differences between expression/complementation in Screen BioInk compared to Endpoint detection. The detected protein clusters formed after expression only with endpoint detection mean that the protein aggregated after expression, thereby lowering the soluble yield. It is evident from the image the presence of speckles for several constructs, indicating the likely presence of protein aggregates. The level of aggregation enables identification of conditions which are worthy of further testing, and identification of conditions having high level of aggregated protein from which further purification is unlikely to give material. Such an experiment is made possible by the high solubility of the modified ccGFP proteins, meaning concentrated droplets of detector can be merged with the expressed protein droplets and the levels of expressed protein measured.

1. A ccGFP variant comprising a mutation at position K45.

2. (canceled)

3. The ccGFP variant according to claim 1 comprising a K45E mutation.

4. The ccGFP variant according to claim 1 which is a ccGFP₁₋₁₀ variant.

5. The ccGFP variant according to claim 1, wherein the ccGFP₁₋₁₀ is complexed with ccGFP₁₁.

6. The ccGFP variant according to claim 4, wherein the ccGFP K45E variant sequence comprises a sequence with greater than 95% homology to sequence having the E fixed:
(SEQ ID NO: 4)
MSMEKQVLKENMKTTYHMDGSVDGHYFEIEGEGTGNPFKGEQELELRVTK
GGPLPFAFDILSPTFTYGNRVFTDYPEDMPDYFKQSLPEGYSWERTMMYE
DGATATASARISLDKNGFVHKSTFHGENFPANGPVMKKKGVDWEPSSETI
TPEDGILKGDVEMFLVLEGGQRLKALFQTTYKANKVVKMPPRHKIEHRLV
RS
or a C or N terminal truncation thereof which binds to ccGFP₁₁ to become fluorescent.

7. The ccGFP variant according to claim 1, wherein the ccGFP K45E variant sequence comprises a sequence:
(SEQ ID NO: 4)
MSMEKQVLKENMKTTYHMDGSVDGHYFEIEGEGTGNPFKGEQELELRVTK
GGPLPFAFDILSPTFTYGNRVFTDYPEDMPDYFKQSLPEGYSWERTMMYE
DGATATASARISLDKNGFVHKSTFHGENFPANGPVMKKKGVDWEPSSETI
TPEDGILKGDVEMFLVLEGGQRLKALFQTTYKANKVVKMPPRHKIEHRLV
RS.

8. The ccGFP variant according to claim 1 which further comprises a solubility enhancing sequence.

9. The ccGFP variant according to claim 8, wherein the solubility enhancing sequence is selected from Glutathione S-Transferase (GST), Small Ubiquitin-like Modifier (SUMO), Maltose Binding Protein (MBP), Fasciola hepatica 8 kDa antigen (FH8), Thioredoxin (TRX), Solubility Enhancing Ubiquitous Tag (SNUT), Seventeen kilodalton protein (SKP), Monomeric bacteriophage T7 orc protein (MOCR), E coli secreted protein A (ESPA), T7 phage tail (P17), metal-binding protein (CUSF) or 53-amino-acid-long N-terminal extension sequence (NEXT).

10. The ccGFP variant according to claim 9 comprising a sequence selected from:
(SEQ ID NO: 55)
MSMEKQVLKENMKTTYHMDGSVDGHYFEIEGEGTGNPFKGEQELELRVTKGGPLP
FAFDILSPTFTYGNRVFTDYPEDMPDYFKQSLPEGYSWERTMMYEDGATATASARI
SLDKNGFVHKSTFHGENFPANGPVMKKKGVDWEPSSETITPEDGILKGDVEMFLVL
EGGQRLKALFQTTYKANKVVKMPPRHKIEHRLVRSGSGAEAAAKEAAAKAGSGM
KNESSTNATNTKQWRDETKGFRDEAKRFKNTAGGGGGSHHHHHH[[.]];
(SEQ ID NO: 56)
MSMEKQVLKENMKTTYHMDGSVDGHYFEIEGEGTGNPFKGEQELELRVTKGGPLP
FAFDILSPTFTYGNRVFTDYPEDMPDYFKQSLPEGYSWERTMMYEDGATATASARI
SLDKNGFVHKSTFHGENFPANGPVMKKKGVDWEPSSETITPEDGILKGDVEMFLVL
EGGQRLKALFQTTYKANKVVKMPPRHKIEHRLVRSGSGAEAAAKEAAAKAGSGM
AVQHSNAPLIDLGAEMKKQHKEAAPEGAAPAQGKAPAAEAKKEEAPKPKPVVGG
GGSHHHHHH[[.]];
(SEQ ID NO: 57)
MSMEKQVLKENMKTTYHMDGSVDGHYFEIEGEGTGNPFKGEQELELRVTKGGPLP
FAFDILSPTFTYGNRVFTDYPEDMPDYFKQSLPEGYSWERTMMYEDGATATASARI
SLDKNGFVHKSTFHGENFPANGPVMKKKGVDWEPSSETITPEDGILKGDVEMFLVL
EGGQRLKALFQTTYKANKVVKMPPRHKIEHRLVRSGSGAEAAAKEAAAKAGSGM
PSVQEVEKLLHVLDRNGDGKVSAEELKAFADDSKCPLDSNKIKAFIKEHDKNKDGK
LDLKELVSILSSGGGGSHHHHHH[[.]];
(SEQ ID NO: 58)
MSMEKQVLKENMKTTYHMDGSVDGHYFEIEGEGTGNPFKGEQELELRVTKGGPLP
FAFDILSPTFTYGNRVFTDYPEDMPDYFKQSLPEGYSWERTMMYEDGATATASARI
SLDKNGFVHKSTFHGENFPANGPVMKKKGVDWEPSSETITPEDGILKGDVEMFLVL
EGGQRLKALFQTTYKANKVVKMPPRHKIEHRLVRSGSGAEAAAKEAAAKAGSGM
SDKIIHLTDDSFDTDVLKADGAILVDFWAEWCGPCKMIAPILDEIADEYQGKLTVAK
LNIDQNPGTAPKYGIRGIPTLLLFKNGEVAATKVGALSKGQLKEFLDANLAGGGGS
HHHHHH[[.]];
(SEQ ID NO: 59)
MSMEKQVLKENMKTTYHMDGSVDGHYFEIEGEGTGNPFKGEQELELRVTKGGPLP
FAFDILSPTFTYGNRVFTDYPEDMPDYFKQSLPEGYSWERTMMYEDGATATASARI
SLDKNGFVHKSTFHGENFPANGPVMKKKGVDWEPSSETITPEDGILKGDVEMFLVL
EGGQRLKALFQTTYKANKVVKMPPRHKIEHRLVRSGSGAEAAAKEAAAKAGSGM
AMSNMTYNNVFDHAYEMLKENIRYDDIRDTDDLHDAIHMAADNAVPHYYADIFS
VMASEGIDLEFEDSGLMPDTKDVIRILQARIYEQLTIDLWEDAEDLLNEYLEEVEEY
EEDEEGGGGSHHHHHH[[.]];
(SEQ ID NO: 60)
MSMEKQVLKENMKTTYHMDGSVDGHYFEIEGEGTGNPFKGEQELELRVTKGGPLP
FAFDILSPTFTYGNRVFTDYPEDMPDYFKQSLPEGYSWERTMMYEDGATATASARI
SLDKNGFVHKSTFHGENFPANGPVMKKKGVDWEPSSETITPEDGILKGDVEMFLVL
EGGQRLKALFQTTYKANKVVKMPPRHKIEHRLVRSGSGAEAAAKEAAAKAGSGM
KPHIDNYLHDKDKDERIEQYDKNVKEQASKDKKQQAKPQIPKDKSKVAGYIEIPDA
DIKEPVYPGPATPEQLNRGVSFAEENESLDDQNISIAGHTFIDRPNYQFTNLKAAKKG
SMVYFKVGNETRKYKMTSIRDVKPTDVEVLDGSGGGGSHHHHHH[[.]];
(SEQ ID NO: 61)
MSMEKQVLKENMKTTYHMDGSVDGHYFEIEGEGTGNPFKGEQELELRVTKGGPLP
FAFDILSPTFTYGNRVFTDYPEDMPDYFKQSLPEGYSWERTMMYEDGATATASARI
SLDKNGFVHKSTFHGENFPANGPVMKKKGVDWEPSSETITPEDGILKGDVEMFLVL
EGGQRLKALFQTTYKANKVVKMPPRHKIEHRLVRSGSGAEAAAKEAAAKAGSGM
SPILGYWKIKGLVQPTRLLLEYLEEKYEEHLYERDEGDKWRNKKFELGLEFPNLPY
YIDGDVKLTQSMAIIRYIADKHNMLGGCPKERAEISMLEGAVLDIRYGVSRIAYSKD
FETLKVDFLSKLPEMLKMFEDRLCHKTYLNGDHVTHPDFMLYDALDVVLYMDPM
CLDAFPKLVCFKKRIEAIPQIDKYLKSSKYIAWPLQGWQATFGGGDHPPKGGGGSH
HHHHH[[.]];
(SEQ ID NO: 62)
MSMEKQVLKENMKTTYHMDGSVDGHYFEIEGEGTGNPFKGEQELELRVTKGGPLP
FAFDILSPTFTYGNRVFTDYPEDMPDYFKQSLPEGYSWERTMMYEDGATATASARI
SLDKNGFVHKSTFHGENFPANGPVMKKKGVDWEPSSETITPEDGILKGDVEMFLVL
EGGQRLKALFQTTYKANKVVKMPPRHKIEHRLVRSGSGAEAAAKEAAAKAGSGM
KIEEGKLVIWINGDKGYNGLAEVGKKFEKDTGIKVTVEHPDKLEEKFPQVAATGDG
PDIIFWAHDRFGGYAQSGLLAEITPDKAFQDKLYPFTWDAVRYNGKLIAYPIAVEAL
SLIYNKDLLPNPPKTWEEIPALDKELKAKGKSALMFNLQEPYFTWPLIAADGGYAF
KYENGKYDIKDVGVDNAGAKAGLTFLVDLIKNKHMNADTDYSIAEAAFNKGETA
MTINGPWAWSNIDTSKVNYGVTVLPTFKGQPSKPFVGVLSAGINAASPNKELAKEF
LENYLLTDEGLEAVNKDKPLGAVALKSYEEELAKDPRIAATMENAQKGEIMPNIPQ
MSAFWYAVRTAVINAASGRQTVDEALKDAQTGGGGSHHHHHH[[.]];
(SEQ ID NO: 63)
MSMEKQVLKENMKTTYHMDGSVDGHYFEIEGEGTGNPFKGEQELELRVTKGGPLP
FAFDILSPTFTYGNRVFTDYPEDMPDYFKQSLPEGYSWERTMMYEDGATATASARI
SLDKNGFVHKSTFHGENFPANGPVMKKKGVDWEPSSETITPEDGILKGDVEMFLVL
EGGQRLKALFQTTYKANKVVKMPPRHKIEHRLVRSGSGAEAAAKEAAAKAGSGM
ANEHHHETMSEAQPQVISATGVVKGIDLESKKITIHHDPIAAVNWPEMTMRFTITPQ
TKMSEIKTGDKVAFNFVQQGNLSLLQDIKVSQGGGGSHHHHHH[[.]];
and
(SEQ ID NO: 64)
MSMEKQVLKENMKTTYHMDGSVDGHYFEIEGEGTGNPFKGEQELELRVTKGGPLP
FAFDILSPTFTYGNRVFTDYPEDMPDYFKQSLPEGYSWERTMMYEDGATATASARI
SLDKNGFVHKSTFHGENFPANGPVMKKKGVDWEPSSETITPEDGILKGDVEMFLVL
EGGQRLKALFQTTYKANKVVKMPPRHKIEHRLVRSGSGAEAAAKEAAAKAGSGM
VDNKFNKEQQNAYYEILHLPNLNEGQRNAFIQSLKDDPSQSANLLAEAKKLNDAQ
APKVDNKFNKEQQNAFYEILHLPNLNEEQRNAFIQSLKDDPSQSANLLAEAEKLND
AQAPKGGGGSHHHHHH.

11. The ccGFP variant according to claim 1, wherein the protein is at a concentration of greater than 10 mg/mL.

12. The ccGFP variant according to claim 1, wherein the protein is at a concentration of greater than 50 mg/mL.

13. A nucleic acid sequence coding for an amino acid sequence according to claim 1.

14. A nucleic acid sequence according to claim 13 which comprises:
(SEQ ID NO: 5)
ATGAGCATGGAAAAACAGGTGCTGAAAGAAAACATGAAAACCACCTATCA
CATGGATGGTAGCGTTGATGGTCACTATTTTGAAATTGAAGGTGAAGGCA
CCGGCAATCCGTTTAAAGGTGAACAAGAACTGGAACTCCGTGTTACCAAA
GGTGGTCCGCTGCCGTTTGCATTTGATATTCTGAGCCCGACCTTTACCTA
TGGTAATCGTGTTTTTACCGACTATCCGGAAGATATGCCGGATTATTTCA
AACAGAGCCTGCCGGAAGGTTATAGCTGGGAACGTACCATGATGTATGAA
GATGGTGCAACCGCAACCGCCAGCGCACGTATTAGCCTGGATAAAAATGG
TTTTGTGCATAAGAGCACCTTTCACGGTGAAAACTTTCCGGCAAATGGTC
CGGTTATGAAAAAGAAAGGTGTTGATTGGGAACCGAGCAGCGAAACCATT
ACACCGGAAGATGGTATTCTGAAAGGTGATGTTGAAATGTTTCTGGTTCT
GGAAGGTGGTCAGCGTCTGAAAGCCCTGTTTCAGACCACCTATAAAGCCA
ATAAAGTGGTTAAAATGCCTCCGCGTCATAAAATTGAACATCGTCTGGTT
CGTAGC.

15. A method of detecting a protein of interest comprising taking a protein of interest attached to ccGFP₁₁, binding the ccGFP₁₁ to a ccGFP₁₋₁₀ variant according to claim 4 and monitoring the presence of the protein of interest by detecting a fluorescent signal from the assembled ccGFP₁₋₁₁.

16. A method for improving the soluble yield of an expressed protein of interest (POI) by expressing the POI in the presence of a ccGFP₁₋₁₀ variant according to claim 4 which binds to a ccGFP₁₁ binding sequence attached to the POI.

17. The method according to claim 15, wherein expression of the protein of interest attached to ccGFP₁₁ is performed in droplets on a digital microfluidic device.

18. The method according to claim 17 comprising expressing a protein attached to ccGFP₁₁ in droplets on a digital microfluidic device in the presence of a ccGFP₁₋₁₀ variant comprising a mutation at position K45 and monitoring the presence of the protein of interest by detecting a fluorescent signal from the assembled ccGFP₁₋₁₁.

19. The method according to claim 17, wherein the digital microfluidic device is an active-matrix thin film transistor (AM-TFT) based device.

20. The method according to claim 17, wherein the digital microfluidic device comprises two parallel plates that are separated by a spacer to define a fluid volume filled with a hydrophobic or non-ionic liquid and the hydrophobic or non-ionic liquid contains surfactant.

21-22. (canceled)

23. The method according to claim 20, wherein hydrophobic or non-ionic liquid is decane, dodecane or dodecamethylpentasiloxane.


SEQUENCE LISTING (78 sequences)

SEQ ID NO: 1 - protein, 221 aa, synthetic construct
MSLSKQVVKEDMKMTYHMDGCVNGHYFTIEGEGTGKPFKGQKTLKLRVTEGGPLPFAFDILSATFTYGNRCFCDYPEDMPDYFKQSLPEGYSWERTMMYEDGACGTASAHISLDKNGFVHNSTFHGVNFPANGPVMKKKGVNWEPSSEKITACDGILKGDVTMFLVLEGGHRLKCLFQTTYKADKVVKMPPNHIIEHRLVRSEDGDAVQIQEHAVAKYFTV

SEQ ID NO: 2 - protein, 221 aa, synthetic construct
MSLSKQVVKEDMKMTYHMDGCVNGHYFTIEGEGTGKPFKGQKTLELRVTEGGPLPFAFDILSATFTYGNRCFCDYPEDMPDYFKQSLPEGYSWERTMMYEDGACGTASAHISLDKNGFVHNSTFHGVNFPANGPVMKKKGVNWEPSSEKITACDGILKGDVTMFLVLEGGHRLKCLFQTTYKADKVVKMPPNHIIEHRLVRSEDGDAVQIQEHAVAKYFTV

SEQ ID NO: 3 - protein, 9 aa, synthetic construct
GEQELELRV

SEQ ID NO: 4 - protein, 202 aa, synthetic construct
MSMEKQVLKENMKTTYHMDGSVDGHYFEIEGEGTGNPFKGEQELELRVTKGGPLPFAFDILSPTFTYGNRVFTDYPEDMPDYFKQSLPEGYSWERTMMYEDGATATASARISLDKNGFVHKSTFHGENFPANGPVMKKKGVDWEPSSETITPEDGILKGDVEMFLVLEGGQRLKALFQTTYKANKVVKMPPRHKIEHRLVRS

SEQ ID NO: 5 - protein, 606 aa, synthetic construct
ATGAGCATGGAAAAACAGGTGCTGAAAGAAAACATGAAAACCACCTATCACATGGATGGTAGCGTTGATGGTCACTATTTTGAAATTGAAGGTGAAGGCACCGGCAATCCGTTTAAAGGTGAACAAGAACTGGAACTCCGTGTTACCAAAGGTGGTCCGCTGCCGTTTGCATTTGATATTCTGAGCCCGACCTTTACCTATGGTAATCGTGTTTTTACCGACTATCCGGAAGATATGCCGGATTATTTCAAACAGAGCCTGCCGGAAGGTTATAGCTGGGAACGTACCATGATGTATGAAGATGGTGCAACCGCAACCGCCAGCGCACGTATTAGCCTGGATAAAAATGGTTTTGTGCATAAGAGCACCTTTCACGGTGAAAACTTTCCGGCAAATGGTCCGGTTATGAAAAAGAAAGGTGTTGATTGGGAACCGAGCAGCGAAACCATTACACCGGAAGATGGTATTCTGAAAGGTGATGTTGAAATGTTTCTGGTTCTGGAAGGTGGTCAGCGTCTGAAAGCCCTGTTTCAGACCACCTATAAAGCCAATAAAGTGGTTAAAATGCCTCCGCGTCATAAAATTGAACATCGTCTGGTTCGTAGC

SEQ ID NO: 6 - protein, 202 aa, synthetic construct
MSMEKQVLKENMKTTYHMDGSVDGHYFEIEGEGTGNPFKGEQELKLRVTKGGPLPFAFDILSPTFTYGNRVFTDYPEDMPDYFKQSLPEGYSWERTMMYEDGATATASARISLDKNGFVHKSTFHGENFPANGPVMKKKGVDWEPSSETITPEDGILKGDVEMFLVLEGGQRLKALFQTTYKANKVVKMPPRHKIEHRLVRS

SEQ ID NO: 7 - protein, 19 aa, synthetic construct
KRDHMVLLEFVTAAGITGT

SEQ ID NO: 8 - protein, 19 aa, synthetic construct
KRDHMVLHEFVTAAGITGT

SEQ ID NO: 9 - protein, 17 aa, synthetic construct
KRDHMVLHESVNAAGIT

SEQ ID NO: 10 - protein, 16 aa, synthetic construct
RDHMVLHEYVNAAGIT

SEQ ID NO: 11 - protein, 17 aa, synthetic construct
GDAVQIQEHAVAKYFTV

SEQ ID NO: 12 - protein, 17 aa, synthetic construct
GDTVQLQEHAVAKYFTV

SEQ ID NO: 13 - protein, 17 aa, synthetic construct
GETIQLQEHAVAKYFTE

SEQ ID NO: 14 - DNA, 51 bp, synthetic construct
  source 1..51: mol_type = other DNA
ggtgataccgttcagctgcaagaacatgcagttgcaaaatactttaccgtg

SEQ ID NO: 15 - DNA, 51 bp, synthetic construct
  source 1..51: mol_type = other DNA
ggtgaaaccatccagttacaagaacacgccgtggccaaatatttcaccgaa

SEQ ID NO: 16 - protein, 9 aa, synthetic construct
GEQELKLRV

SEQ ID NO: 17 - protein, 9 aa, synthetic construct
HKIEHRLVR

SEQ ID NO: 18 - protein, 8 aa, synthetic construct
LQEHAVAK

SEQ ID NO: 19 - DNA, 24 bp, synthetic construct
  source 1..24: mol_type = other DNA
ttacaagaacacgccgtggccaaa

SEQ ID NO: 20 - protein, 13 aa, synthetic construct
SRLEEELRRRLTE

SEQ ID NO: 21 - protein, 15 aa, synthetic construct
GLNDIFEAQKIEWHE

SEQ ID NO: 22 - protein, 4 aa, synthetic construct
EPEA

SEQ ID NO: 23 - protein, 26 aa, Unidentified
KRRWKKNFIAVSAANRFKKISSSGAL

SEQ ID NO: 24 - protein, 23 aa, synthetic construct
DIPATYEFTDGKHYITNEPIPPK

SEQ ID NO: 25 - protein, 13 aa, synthetic construct
GAPVPYPDPLEPR

SEQ ID NO: 26 - protein, 8 aa, synthetic construct
DYKDDDDK

SEQ ID NO: 27 - protein, 19 aa, synthetic construct
EELLSKNYHLENEVARLKK

SEQ ID NO: 28 - protein, 9 aa, Human Influenza Virus
YPYDVPDYA

SEQ ID NO: 29 - protein, 6 aa, synthetic construct
HHHHHH

SEQ ID NO: 30 - protein, 16 aa, Streptococcus pyogenes
TDKDMTITFTNKKDAE

SEQ ID NO: 31 - protein, 20 aa, synthetic construct
FIDTNNDGWIEGDELLLEEG

SEQ ID NO: 32 - protein, 10 aa, Homo sapiens
EQKLISEEDL

SEQ ID NO: 33 - protein, 18 aa, synthetic construct
TKENPRSNQEESYDDNES

SEQ ID NO: 34 - protein, 7 aa, synthetic construct
EEEEEEE

SEQ ID NO: 35 - protein, 7 aa, synthetic construct
RRRRRRR

SEQ ID NO: 36 - protein, 9 aa, Bos taurus
TETSQVAPA

SEQ ID NO: 37 - protein, 38 aa, synthetic construct
MDEKTTGWRGGHVVEGLAGELEQLRARLEHHPQGQREP

SEQ ID NO: 38 - protein, 13 aa, synthetic construct
DPIVMIDNDKPIT

SEQ ID NO: 39 - protein, 13 aa, Unidentified
STVPVAPPRRRRG

SEQ ID NO: 40 - protein, 5 aa, synthetic construct
GSHHW

SEQ ID NO: 41 - protein, 12 aa, synthetic construct
KLGDIEFIKVNK

SEQ ID NO: 42 - protein, 13 aa, Escherichia coli
SLAELLNAGLGGS

SEQ ID NO: 43 - protein, 8 aa, Homo sapiens
TQDPSRVG

SEQ ID NO: 44 - protein, 12 aa, synthetic construct
PDRVRAVSHWSS

SEQ ID NO: 45 - protein, 13 aa, Streptococcus pyogenes
AHIVMVDAYKPTK

SEQ ID NO: 46 - protein, 15 aa, Unidentified
KETAAAKFERQHMDS

SEQ ID NO: 47 - protein, 9 aa, synthetic construct
AWAHPQPGG

SEQ ID NO: 48 - protein, 9 aa, synthetic construct
AWRHPQFGG

SEQ ID NO: 49 - protein, 8 aa, synthetic construct
WSHPQFEK

SEQ ID NO: 50 - protein, 11 aa, synthetic construct
MASMTGGQQMG

SEQ ID NO: 51 - protein, 10 aa, synthetic construct
EVHTNQDPLD

SEQ ID NO: 52 - protein, 6 aa, synthetic construct
CCPGCC

SEQ ID NO: 53 - protein, 11 aa, vesicular stomatitis virus
YTDIEMNRLGK

SEQ ID NO: 54 - protein, 8 aa, synthetic construct
DLYDDDDK

SEQ ID NO: 55 - protein, 193 aa, synthetic construct
MSMEKQVLKENMKTTYHMDGSVDGHYFEIEGEGTGNPFKGEQELELRVTKGGPLPFAFDILSPTFTYGNRVFTDYPEDMPDYFKQSLPEGYSWERTMMYEDGATATASARISLDKNGFVHKSTFHGENFPANGPVMKKKGVDWEPSSETITPEDGILKGDVEMFLVLEGGQRLKALFQTTYKANKVVKMPPRH

SEQ ID NO: 56 - protein, 284 aa, synthetic construct
MSMEKQVLKENMKTTYHMDGSVDGHYFEIEGEGTGNPFKGEQELELRVTKGGPLPFAFDILSPTFTYGNRVFTDYPEDMPDYFKQSLPEGYSWERTMMYEDGATATASARISLDKNGFVHKSTFHGENFPANGPVMKKKGVDWEPSSETITPEDGILKGDVEMFLVLEGGQRLKALFQTTYKANKVVKMPPRHKIEHRLVRSGSGAEAAAKEAAAKAGSGMAVQHSNAPLIDLGAEMKKQHKEAAPEGAAPAQGKAPAAEAKKEEAPKPKPVVGGGGSHHHHHH

SEQ ID NO: 57 - protein, 300 aa, synthetic construct
MSMEKQVLKENMKTTYHMDGSVDGHYFEIEGEGTGNPFKGEQELELRVTKGGPLPFAFDILSPTFTYGNRVFTDYPEDMPDYFKQSLPEGYSWERTMMYEDGATATASARISLDKNGFVHKSTFHGENFPANGPVMKKKGVDWEPSSETITPEDGILKGDVEMFLVLEGGQRLKALFQTTYKANKVVKMPPRHKIEHRLVRSGSGAEAAAKEAAAKAGSGMPSVQEVEKLLHVLDRNGDGKVSAEELKAFADDSKCPLDSNKIKAFIKEHDKNKDGKLDLKELVSILSSGGGGSHHHHHH

SEQ ID NO: 58 - protein, 340 aa, synthetic construct
MSMEKQVLKENMKTTYHMDGSVDGHYFEIEGEGTGNPFKGEQELELRVTKGGPLPFAFDILSPTFTYGNRVFTDYPEDMPDYFKQSLPEGYSWERTMMYEDGATATASARISLDKNGFVHKSTFHGENFPANGPVMKKKGVDWEPSSETITPEDGILKGDVEMFLVLEGGQRLKALFQTTYKANKVVKMPPRHKIEHRLVRSGSGAEAAAKEAAAKAGSGMSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGGGGSHHHHHH

SEQ ID NO: 59 - protein, 348 aa, synthetic construct
MSMEKQVLKENMKTTYHMDGSVDGHYFEIEGEGTGNPFKGEQELELRVTKGGPLPFAFDILSPTFTYGNRVFTDYPEDMPDYFKQSLPEGYSWERTMMYEDGATATASARISLDKNGFVHKSTFHGENFPANGPVMKKKGVDWEPSSETITPEDGILKGDVEMFLVLEGGQRLKALFQTTYKANKVVKMPPRHKIEHRLVRSGSGAEAAAKEAAAKAGSGMAMSNMTYNNVFDHAYEMLKENIRYDDIRDTDDLHDAIHMAADNAVPHYYADIFSVMASEGIDLEFEDSGLMPDTKDVIRILQARIYEQLTIDLWEDAEDLLNEYLEEVEEYEEDEEGGGGSHHHHHH

SEQ ID NO: 60 - protein, 379 aa, synthetic construct
MSMEKQVLKENMKTTYHMDGSVDGHYFEIEGEGTGNPFKGEQELELRVTKGGPLPFAFDILSPTFTYGNRVFTDYPEDMPDYFKQSLPEGYSWERTMMYEDGATATASARISLDKNGFVHKSTFHGENFPANGPVMKKKGVDWEPSSETITPEDGILKGDVEMFLVLEGGQRLKALFQTTYKANKVVKMPPRHKIEHRLVRSGSGAEAAAKEAAAKAGSGMKPHIDNYLHDKDKDERIEQYDKNVKEQASKDKKQQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVEVLDGSGGGGSHHHHHH

SEQ ID NO: 61 - protein, 449 aa, synthetic construct
MSMEKQVLKENMKTTYHMDGSVDGHYFEIEGEGTGNPFKGEQELELRVTKGGPLPFAFDILSPTFTYGNRVFTDYPEDMPDYFKQSLPEGYSWERTMMYEDGATATASARISLDKNGFVHKSTFHGENFPANGPVMKKKGVDWEPSSETITPEDGILKGDVEMFLVLEGGQRLKALFQTTYKANKVVKMPPRHKIEHRLVRSGSGAEAAAKEAAAKAGSGMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKGGGGSHHHHHH

SEQ ID NO: 62 - protein, 598 aa, synthetic construct
MSMEKQVLKENMKTTYHMDGSVDGHYFEIEGEGTGNPFKGEQELELRVTKGGPLPFAFDILSPTFTYGNRVFTDYPEDMPDYFKQSLPEGYSWERTMMYEDGATATASARISLDKNGFVHKSTFHGENFPANGPVMKKKGVDWEPSSETITPEDGILKGDVEMFLVLEGGQRLKALFQTTYKANKVVKMPPRHKIEHRLVRSGSGAEAAAKEAAAKAGSGMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGGGGSHHHHHH

SEQ ID NO: 63 - protein, 321 aa, synthetic construct
MSMEKQVLKENMKTTYHMDGSVDGHYFEIEGEGTGNPFKGEQELELRVTKGGPLPFAFDILSPTFTYGNRVFTDYPEDMPDYFKQSLPEGYSWERTMMYEDGATATASARISLDKNGFVHKSTFHGENFPANGPVMKKKGVDWEPSSETITPEDGILKGDVEMFLVLEGGQRLKALFQTTYKANKVVKMPPRHKIEHRLVRSGSGAEAAAKEAAAKAGSGMANEHHHETMSEAQPQVISATGVVKGIDLESKKITIHHDPIAAVNWPEMTMRFTITPQTKMSEIKTGDKVAFNFVQQGNLSLLQDIKVSQGGGGSHHHHHH

SEQ ID NO: 64 - protein, 348 aa, synthetic construct
MSMEKQVLKENMKTTYHMDGSVDGHYFEIEGEGTGNPFKGEQELELRVTKGGPLPFAFDILSPTFTYGNRVFTDYPEDMPDYFKQSLPEGYSWERTMMYEDGATATASARISLDKNGFVHKSTFHGENFPANGPVMKKKGVDWEPSSETITPEDGILKGDVEMFLVLEGGQRLKALFQTTYKANKVVKMPPRHKIEHRLVRSGSGAEAAAKEAAAKAGSGMVDNKFNKEQQNAYYEILHLPNLNEGQRNAFIQSLKDDPSQSANLLAEAKKLNDAQAPKVDNKFNKEQQNAFYEILHLPNLNEEQRNAFIQSLKDDPSQSANLLAEAEKLNDAQAPKGGGGSHHHHHH

SEQ ID NO: 65 - DNA, 1797 bp, synthetic construct
  source 1..1797: mol_type = other DNA
atgagcatggaaaaacaggtgctgaaagaaaacatgaaaaccacctatcacatggatggtagcgttgatggtcactattttgaaattgaaggtgaaggcaccggcaatccgtttaaaggtgaacaagaactggaactccgtgttaccaaaggtggtccgctgccgtttgcatttgatattctgagcccgacctttacctatggtaatcgtgtttttaccgactatccggaagatatgccggattatttcaaacagagcctgccggaaggttatagctgggaacgtaccatgatgtatgaagatggtgcaaccgcaaccgccagcgcacgtattagcctggataaaaatggttttgtgcataagagcacctttcacggtgaaaactttccggcaaatggtccggttatgaaaaagaaaggtgttgattgggaaccgagcagcgaaaccattacaccggaagatggtattctgaaaggtgatgttgaaatgtttctggttctggaaggtggtcagcgtctgaaagccctgtttcagaccacctataaagccaataaagtggttaaaatgcctccgcgtcataaaattgaacatcgtctggttcgtagcggtagtggtgcagaagctgctgcaaaagaggcagctgcgaaggcaggtagtggtatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactggcggtggcggtagccatcaccaccatcatcactaa

SEQ ID NO: 66 - DNA, 798 bp, synthetic construct
  source 1..798: mol_type = other DNA
atgagcatggaaaaacaggtgctgaaagaaaacatgaaaaccacctatcacatggatggtagcgttgatggtcactattttgaaattgaaggtgaaggcaccggcaatccgtttaaaggtgaacaagaactggaactccgtgttaccaaaggtggtccgctgccgtttgcatttgatattctgagcccgacctttacctatggtaatcgtgtttttaccgactatccggaagatatgccggattatttcaaacagagcctgccggaaggttatagctgggaacgtaccatgatgtatgaagatggtgcaaccgcaaccgccagcgcacgtattagcctggataaaaatggttttgtgcataagagcacctttcacggtgaaaactttccggcaaatggtccggttatgaaaaagaaaggtgttgattgggaaccgagcagcgaaaccattacaccggaagatggtattctgaaaggtgatgttgaaatgtttctggttctggaaggtggtcagcgtctgaaagccctgtttcagaccacctataaagccaataaagtggttaaaatgcctccgcgtcataaaattgaacatcgtctggttcgtagcggtagtggtgcagaagctgctgcaaaagaggcagctgcgaaggcaggtagtggtatgaagaacgagagcagcacaaatgcgacaaataccaagcagtggcgcgacgagaccaagggtttccgcgacgaggcaaaacgtttcaaaaacactgcgggaggcggtggcggtagccatcaccaccatcatcactaa

SEQ ID NO: 67 - DNA, 855 bp, synthetic construct
  source 1..855: mol_type = other DNA
atgagcatggaaaaacaggtgctgaaagaaaacatgaaaaccacctatcacatggatggtagcgttgatggtcactattttgaaattgaaggtgaaggcaccggcaatccgtttaaaggtgaacaagaactggaactccgtgttaccaaaggtggtccgctgccgtttgcatttgatattctgagcccgacctttacctatggtaatcgtgtttttaccgactatccggaagatatgccggattatttcaaacagagcctgccggaaggttatagctgggaacgtaccatgatgtatgaagatggtgcaaccgcaaccgccagcgcacgtattagcctggataaaaatggttttgtgcataagagcacctttcacggtgaaaactttccggcaaatggtccggttatgaaaaagaaaggtgttgattgggaaccgagcagcgaaaccattacaccggaagatggtattctgaaaggtgatgttgaaatgtttctggttctggaaggtggtcagcgtctgaaagccctgtttcagaccacctataaagccaataaagtggttaaaatgcctccgcgtcataaaattgaacatcgtctggttcgtagcggtagtggtgcagaagctgctgcaaaagaggcagctgcgaaggcaggtagtggtatggccgtacagcatagtaacgctcctttaatcgacctgggtgcagaaatgaagaaacaacataaagaggccgcgccagagggtgccgcgcctgcacaaggaaaggccccagcagcagaggcaaaaaaagaggaagctccaaaacccaaaccggtcgtgggcggtggcggtagccatcaccaccatcatcactaa

SEQ ID NO: 68 - DNA, 903 bp, synthetic construct
  source 1..903: mol_type = other DNA
atgagcatggaaaaacaggtgctgaaagaaaacatgaaaaccacctatcacatggatggtagcgttgatggtcactattttgaaattgaaggtgaaggcaccggcaatccgtttaaaggtgaacaagaactggaactccgtgttaccaaaggtggtccgctgccgtttgcatttgatattctgagcccgacctttacctatggtaatcgtgtttttaccgactatccggaagatatgccggattatttcaaacagagcctgccggaaggttatagctgggaacgtaccatgatgtatgaagatggtgcaaccgcaaccgccagcgcacgtattagcctggataaaaatggttttgtgcataagagcacctttcacggtgaaaactttccggcaaatggtccggttatgaaaaagaaaggtgttgattgggaaccgagcagcgaaaccattacaccggaagatggtattctgaaaggtgatgttgaaatgtttctggttctggaaggtggtcagcgtctgaaagccctgtttcagaccacctataaagccaataaagtggttaaaatgcctccgcgtcataaaattgaacatcgtctggttcgtagcggtagtggtgcagaagctgctgcaaaagaggcagctgcgaaggcaggtagtggtatgcccagtgtacaagaagtagaaaaactattacatgttctagataggaatggagacggcaaggtgtctgccgaagaattaaaagcatttgctgacgattccaaatgtcctttggactcaaataaaattaaagcttttataaaagaacatgataaaaataaggatggtaaacttgatttaaaagagcttgtaagtattttgtcatctggcggtggcggtagccatcaccaccatcatcactaa

SEQ ID NO: 69 - DNA, 1797 bp, synthetic construct; identical to SEQ ID NO: 65
  source 1..1797: mol_type = other DNA

SEQ ID NO: 70 - DNA, 1023 bp, synthetic construct
  source 1..1023: mol_type = other DNA
atgagcatggaaaaacaggtgctgaaagaaaacatgaaaaccacctatcacatggatggtagcgttgatggtcactattttgaaattgaaggtgaaggcaccggcaatccgtttaaaggtgaacaagaactggaactccgtgttaccaaaggtggtccgctgccgtttgcatttgatattctgagcccgacctttacctatggtaatcgtgtttttaccgactatccggaagatatgccggattatttcaaacagagcctgccggaaggttatagctgggaacgtaccatgatgtatgaagatggtgcaaccgcaaccgccagcgcacgtattagcctggataaaaatggttttgtgcataagagcacctttcacggtgaaaactttccggcaaatggtccggttatgaaaaagaaaggtgttgattgggaaccgagcagcgaaaccattacaccggaagatggtattctgaaaggtgatgttgaaatgtttctggttctggaaggtggtcagcgtctgaaagccctgtttcagaccacctataaagccaataaagtggttaaaatgcctccgcgtcataaaattgaacatcgtctggttcgtagcggtagtggtgcagaagctgctgcaaaagaggcagctgcgaaggcaggtagtggtatgtcagataaaataattcatttaacagatgatagttttgatactgatgtattgaaagcagatggagctatcctcgttgatttttgggctgaatggtgtggaccctgtaaaatgattgcacctattttagatgaaattgctgatgaatatcaaggtaaattaacagtcgctaaattaaatattgatcaaaatccaggtactgctccaaaatatggaattagaggaatacctactcttttattatttaaaaatggcgaagtggctgcaacaaaagtgggagctttatctaaaggtcaactaaaagaatttttagatgcaaatcttgcaggcggtggcggtagccatcaccaccatcatcactaa

SEQ ID NO: 71 - DNA, 1047 bp, synthetic construct
  source 1..1047: mol_type = other DNA
atgagcatggaaaaacaggtgctgaaagaaaacatgaaaaccacctatcacatggatggtagcgttgatggtcactattttgaaattgaaggtgaaggcaccggcaatccgtttaaaggtgaacaagaactggaactccgtgttaccaaaggtggtccgctgccgtttgcatttgatattctgagcccgacctttacctatggtaatcgtgtttttaccgactatccggaagatatgccggattatttcaaacagagcctgccggaaggttatagctgggaacgtaccatgatgtatgaagatggtgcaaccgcaaccgccagcgcacgtattagcctggataaaaatggttttgtgcataagagcacctttcacggtgaaaactttccggcaaatggtccggttatgaaaaagaaaggtgttgattgggaaccgagcagcgaaaccattacaccggaagatggtattctgaaaggtgatgttgaaatgtttctggttctggaaggtggtcagcgtctgaaagccctgtttcagaccacctataaagccaataaagtggttaaaatgcctccgcgtcataaaattgaacatcgtctggttcgtagcggtagtggtgcagaagctgctgcaaaagaggcagctgcgaaggcaggtagtggtatggcaatgtctaatatgacctataataatgtttttgatcatgcatatgagatgttaaaagaaaacataagatatgatgatattagagacacagatgatcttcatgatgctattcatatggcagcagataacgcagttcctcattattatgctgatatttttagtgtcatggcatcagaaggcattgatctagagtttgaagattctgggcttatgccagatacgaaagacgtaataaggatattacaagcaagaatttacgaacaattaactatagatttatgggaagatgctgaagacttgttaaatgaatatttagaagaagtggaggaatacgaagaagatgaagaaggcggtggcggtagccatcaccaccatcatcactaa

SEQ ID NO: 72 - DNA, 1140 bp, synthetic construct
  source 1..1140: mol_type = other DNA
atgagcatggaaaaacaggtgctgaaagaaaacatgaaaaccacctatcacatggatggtagcgttgatggtcactattttgaaattgaaggtgaaggcaccggcaatccgtttaaaggtgaacaagaactggaactccgtgttaccaaaggtggtccgctgccgtttgcatttgatattctgagcccgacctttacctatggtaatcgtgtttttaccgactatccggaagatatgccggattatttcaaacagagcctgccggaaggttatagctgggaacgtaccatgatgtatgaagatggtgcaaccgcaaccgccagcgcacgtattagcctggataaaaatggttttgtgcataagagcacctttcacggtgaaaactttccggcaaatggtccggttatgaaaaagaaaggtgttgattgggaaccgagcagcgaaaccattacaccggaagatggtattctgaaaggtgatgttgaaatgtttctggttctggaaggtggtcagcgtctgaaagccctgtttcagaccacctataaagccaataaagtggttaaaatgcctccgcgtcataaaattgaacatcgtctggttcgtagcggtagtggtgcagaagctgctgcaaaagaggcagctgcgaaggcaggtagtggtatgaagcctcacattgacaattatttacacgacaaggacaaagacgagcgcattgagcaatacgacaaaaatgtcaaggaacaagcaagcaaggacaagaaacagcaggcgaagcctcaaatccccaaggacaaaagtaaagtcgctgggtacatcgagatcccggatgcagacattaaggagcccgtctatcctggacctgcaacacctgagcagcttaatcgcggggtgtcgtttgcggaagaaaatgagtcgttggacgaccagaatatcagcatcgcaggccacacctttatcgaccgccccaattatcagttcacaaacttgaaagcggccaagaagggttcaatggtatatttcaaggttggaaatgagacgcgcaaatacaagatgacaagtatccgtgacgtcaaacctactgacgtagaagtacttgatggttccggcggtggcggtagccatcaccaccatcatcactaa

SEQ ID NO: 73 - DNA, 1350 bp, synthetic construct
  source 1..1350: mol_type = other DNA
atgagcatggaaaaacaggtgctgaaagaaaacatgaaaaccacctatcacatggatggtagcgttgatggtcactattttgaaattgaaggtgaaggcaccggcaatccgtttaaaggtgaacaagaactggaactccgtgttaccaaaggtggtccgctgccgtttgcatttgatattctgagcccgacctttacctatggtaatcgtgtttttaccgactatccggaagatatgccggattatttcaaacagagcctgccggaaggttatagctgggaacgtaccatgatgtatgaagatggtgcaaccgcaaccgccagcgcacgtattagcctggataaaaatggttttgtgcataagagcacctttcacggtgaaaactttccggcaaatggtccggttatgaaaaagaaaggtgttgattgggaaccgagcagcgaaaccattacaccggaagatggtattctgaaaggtgatgttgaaatgtttctggttctggaaggtggtcagcgtctgaaagccctgtttcagaccacctataaagccaataaagtggttaaaatgcctccgcgtcataaaattgaacatcgtctggttcgtagcggtagtggtgcagaagctgctgcaaaagaggcagctgcgaaggcaggtagtggtatgtccccaatactaggttattggaaaattaaaggccttgttcaacccactcgactattattggaatatcttgaagaaaaatatgaagaacatttgtatgaacgcgatgaaggtgataaatggagaaataaaaaatttgaattgggattggaatttccaaatttaccttattatattgatggtgatgtaaaattaacacaatctatggcaataattcgttatatagctgacaagcacaacatgttgggtggttgtccaaaagaaagagcagaaatttcaatgttagaaggagcggttctagatattagatacggtgtttctagaattgcatatagtaaagactttgaaactttaaaagttgattttcttagcaagttgcctgaaatgctgaaaatgttcgaagataggttatgtcataaaacatatttaaatggtgatcacgtaacccatcctgactttatgttatatgatgctcttgatgttgttttatatatggacccaatgtgtctggatgcttttccaaaattagtttgttttaaaaaaaggattgaagctattcctcaaatagataaatatttaaaaagcagtaaatatatagcttggccacttcagggatggcaagcaacctttggtggtggagatcatcctccaaaaggcggtggcggtagccatcaccaccatcatcactaa

SEQ ID NO: 74 - DNA, 966 bp, synthetic construct
  source 1..966: mol_type = other DNA
atgagcatggaaaaacaggtgctgaaagaaaacatgaaaaccacctatcacatggatggtagcgttgatggtcactattttgaaattgaaggtgaaggcaccggcaatccgtttaaaggtgaacaagaactggaactccgtgttaccaaaggtggtccgctgccgtttgcatttgatattctgagcccgacctttacctatggtaatcgtgtttttaccgactatccggaagatatgccggattatttcaaacagagcctgccggaaggttatagctgggaacgtaccatgatgtatgaagatggtgcaaccgcaaccgccagcgcacgtattagcctggataaaaatggttttgtgcataagagcacctttcacggtgaaaactttccggcaaatggtccggttatgaaaaagaaaggtgttgattgggaaccgagcagcgaaaccattacaccggaagatggtattctgaaaggtgatgttgaaatgtttctggttctggaaggtggtcagcgtctgaaagccctgtttcagaccacctataaagccaataaagtggttaaaatgcctccgcgtcataaaattgaacatcgtctggttcgtagcggtagtggtgcagaagctgctgcaaaagaggcagctgcgaaggcaggtagtggtatggctaacgaacatcatcatgaaaccatgagcgaagcacaaccacaggttattagcgccactggcgtggtaaagggtatcgatctggaaagcaaaaaaatcaccatccatcacgatccgattgctgccgtgaactggccggagatgaccatgcgctttaccatcaccccgcagacgaaaatgagtgaaattaaaaccggcgacaaagtggcgtttaattttgtccagcagggcaacctttctttattacaggatattaaagtcagccagggcggtggcggtagccatcaccaccatcatcactaa

SEQ ID NO: 75 - DNA, 1047 bp, synthetic construct
  source 1..1047: mol_type = other DNA
atgagcatggaaaaacaggtgctgaaagaaaacatgaaaaccacctatcacatggatggtagcgttgatggtcactattttgaaattgaaggtgaaggcaccggcaatccgtttaaaggtgaacaagaactggaactccgtgttaccaaaggtggtccgctgccgtttgcatttgatattctgagcccgacctttacctatggtaatcgtgtttttaccgactatccggaagatatgccggattatttcaaacagagcctgccggaaggttatagctgggaacgtaccatgatgtatgaagatggtgcaaccgcaaccgccagcgcacgtattagcctggataaaaatggttttgtgcataagagcacctttcacggtgaaaactttccggcaaatggtccggttatgaaaaagaaaggtgttgattgggaaccgagcagcgaaaccattacaccggaagatggtattctgaaaggtgatgttgaaatgtttctggttctggaaggtggtcagcgtctgaaagccctgtttcagaccacctataaagccaataaagtggttaaaatgcctccgcgtcataaaattgaacatcgtctggttcgtagcggtagtggtgcagaagctgctgcaaaagaggcagctgcgaaggcaggtagtggtatggttgataacaaattcaataaagaacagcaaaacgcatattacgagattcttcatctgccgaatttgaatgaaggccaacgtaatgcgtttatccagtcccttaaagacgatccttcccagtctgcgaacttgttagcggaggccaaaaaattaaacgatgcccaagctcccaaggtggataataagttcaataaggaacaacagaatgctttttacgaaatcttgcacctgcccaatcttaacgaagaacaacgcaatgctttcattcaaagtctgaaagacgatccctcgcaaagtgcgaacttattggccgaggctgagaaacttaatgacgctcaagcgcccaagggcggtggcggtagccatcaccaccatcatcactaa

SEQ ID NO: 76 - protein, 12 aa, synthetic construct
LQEHAVAKYFTE

SEQ ID NO: 77 - protein, 13 aa, synthetic construct
GETIQLQEHAVAK

SEQ ID NO: 78 - protein, 11 aa, synthetic construct
IQLQEHAVAKY